(19) United States
(12) Patent Application Publication
(10) Pub. No.: US 2025/0052748 A1
(43) Pub. Date: Feb. 13, 2025

(54) CD83 AND ALLO- AND AUTOIMMUNE CONDITIONS

(71) Applicants: REGENTS OF THE UNIVERSITY OF MINNESOTA, Minneapolis, MN (US); H. Lee Moffitt Cancer Center and Research Institute, Inc., Tampa, FL (US)

(72) Inventors: Brian Betts, Minneapolis, MN (US); Shernan Holtan, Minneapolis, MN (US); Marco Davila, Tampa, FL (US)

(21) Appl. No.: 18/719,814

(22) PCT Filed: Dec. 21, 2022
(86) PCT No.: PCT/US22/82106

§ 371 Date: Jun. 13, 2024

Related U.S. Application Data
(60) Provisional application No. 63/292,858, filed on Dec. 22, 2021.

(51) Int. Cl. G01N 33/564; A61K 39/00; C12Q 1/6883

(52) U.S. Cl. CPC G01N 33/564; A61K 39/4611; A61K 39/4631; A61K 39/46433; C12Q 1/6883; C12Q 2600/158; G01N 2333/70503; G01N 2800/245; G01N 2800/50

(57) ABSTRACT

The disclosure provides a method of treating or reducing the risk of developing an alloimmune condition or an autoimmune condition in a subject in need thereof. The method comprises (a) measuring CD83 in a population of immune cells from the subject, and (b) administering to the subject a CD83-targeted therapeutic.

### CROSS-REFERENCE TO RELATED APPLICATIONS

This application claims priority to U.S. Provisional Patent Application No. 63/292,858, filed Dec. 22, 2021, the disclosure of which is hereby incorporated by reference in its entirety.

### FIELD

The application relates generally to materials and methods for identifying and/or treating subjects at risk of suffering from an allo- or autoimmune condition, such as Graft versus Host Disease (GvHD).

### INCORPORATION BY REFERENCE OF MATERIAL SUBMITTED ELECTRONICALLY

A Sequence Listing, which is a part of the present disclosure, is submitted concurrently with the specification as a text file. The name of the text file containing the Sequence Listing is “57191_Seqlisting.XML.” The Sequence Listing was created on Nov. 7, 2022, and is 4,037,579 bytes in size. The subject matter of the Sequence Listing is incorporated by reference herein in its entirety.

### BACKGROUND

Autoimmunity is an immune response to self-antigens, which results in the body attacking normal cells and tissues. There are more than 80 types of autoimmune diseases that present unique clinical symptoms and affect different parts of the body. In contrast, alloimmunity is an immune response to antigens from different members of the same species. Alloimmunity is triggered primarily by differences in the major histocompatibility complex between individuals within a species. The body's response to alloimmune conditions and autoimmune conditions mimics the response against infection, involving activation of immune cells, inflammation, and tissue damage.

Host-anti-graft immune response remains a significant challenge in transplantation settings. Graft versus Host Disease (GvHD) occurs when donor immune cells transferred to an allogeneic recipient attack tissues in the recipient. The skin, liver, and intestinal tract are often affected, resulting in significant damage and morbidity. GvHD is a common complication of allogeneic hematopoietic cell transplantation (HCT), but also may develop after solid organ transplantation. It has been estimated that acute GvHD develops in 35-50% of patients given HLA-identical bone marrow grafts within the first 100 days of transplantation, and chronic GvHD affects about 45% of all long-term survivors. Deeg & Storb, Annu Rev Med. 1984; 35:11-24.

Currently, GvHD is typically treated with immunosuppressive and cytotoxic drugs. A challenge in the field is that most patients are treated prophylactically to inhibit or prevent the onset of GvHD, resulting in many patients being subjected to immune suppressing therapies or cytotoxic therapies unnecessarily or for a longer period of time than needed. There is a need in the art of improved materials and methods for identifying subjects at risk of suffering from allo- or autoimmune conditions prior to the onset of symptoms.

### SUMMARY

The disclosure provides a method of treating or reducing the risk of developing an autoimmune condition in a subject in need thereof. The method comprises (a) measuring CD83 in a population of immune cells from the subject, and (b) administering to the subject a CD83-targeted therapeutic. In one aspect, the autoimmune condition is acute graft-versus-host disease (GvHD) and the population of immune cells in step (a) comprises CD4+ T cells. Optionally, the method is performed within 30 days of the subject receiving allogeneic hematopoietic cell transplantation and, in various aspects, prior to the onset of acute GvHD symptoms. Alternatively, the condition is chronic GvHD and the population of immune cells in step (a) comprises CD19+ B cells and/or T helper follicular cells. In this respect, the method is optionally performed after 80 days of the subject receiving allogeneic hematopoietic cell transplantation and, in various aspects, prior to the onset of chronic GvHD symptoms.

The disclosure further provides a method of identifying a subject at risk of developing acute GvHD. The method comprises measuring CD83 in a population of CD4+ T cells from the subject, wherein the presence of CD83 expression in at least 40% of CD4+ T cells in the population or a gMFI of at least 740.5 in the population of CD4+ T cells indicates that the subject is at risk of developing acute GvHD.

The disclosure also provides a method of identifying a subject at risk of developing chronic GvHD. The method comprises measuring CD83 in a population of CD19+ B cells from the subject, wherein the presence of CD83 expression in at least 19% of CD19+ B cells in the population or a gMFI of at least 398 indicates that the subject is at risk of developing chronic GvHD. Alternatively (or in addition), the method comprises measuring CD83 in a population of T helper follicular cells from the subject, wherein the presence of CD83 expression in at least 25% of T helper follicular cells in the population or a gMFI of at least 489 indicates that the subject is at risk of developing chronic GvHD.

### BRIEF DESCRIPTION OF THE DRAWINGS

FIGS. 1A-1D: CD83 expression on CD4+ T cells is increased upon acute GvHD onset. CD83 expression was evaluated among patients post allogeneic hematopoietic cell transplantation without or with grade II-IV (n=24) acute GVHD (time-matched controls). FIGS. 1A-1D are bar graphs and ROC (receiver operating characteristic curve) depicting % CD83 expression and geometric mean fluorescence intensity are shown. ***0.0001, ****P<0.0001, and AUC=area under the curve. FIG. 1A is a bar graph illustrating % CD83+ CD4+ T cells in subjects not suffering from acute GvHD and subjects suffering from Grade II-IV acute GvHD. FIG. 1C is a bar graph illustrating CD83 gMFI (geometric mean fluorescence intensity) by CD4+ T cells in subjects not suffering from acute GvHD and subjects suffering from Grade II-IV acute GvHD. FIGS. 1B and 1D are a line graphs illustrating % sensitivity.

FIGS. 2A-2B: Earlier acute GvHD diagnosis is associated with increased CD4+ T cell CD83 expression. The figures are line graphs showing the association (Spearman r) between CD4+ T cell CD83 expression and timing of acute GVHD onset using (FIG. 1A) % CD83 expression and (FIG. 1B) gMFI. n=24 grade II-IV acute GVHD patients.

FIGS. 3A-3B: Increased CD4+ T cell CD83 expression by day +100 is associated with reduced survival after alloHCT. FIG. 3A is a line graph showing the overall survival of alloHCT recipients (n=40) based on CD4+ T cell CD83 expression. CD83 expression was determined at time of acute GVHD diagnosis (n=20) or sampled among time-matched controls (n=20). A gMFI cutoff of 740.5 was used to determine low versus high CD83 expression. Top line—low CD83 gMFI expression; bottom line—high CD83 gMFI expression. FIG. 3B is a line graph showing the overall survival of the alloHCT recipients based on diagnosis of grade II-IV acute GVHD by day +100. Top line—no GvHD; bottom line—Grade II-IV.

FIGS. 4A-4H: CD83 expression on B cells and T helper follicular cells is increased upon chronic GVHD onset. CD83 expression was evaluated among patients post allogeneic hematopoietic cell transplantation without (n=24) or with chronic GVHD (n=24) (time-matched controls). FIGS. 4A and 4D illustrate % CD83+ cells (FIG. 4A) and CD83 gMFI (geometric mean fluorescence intensity; FIG. 4D) on B cells, T helper follicular cells (Tfh) and monocytes. FIGS. 4B, 4C, 4E, and 4F are ROC curves depicting % CD83 expression and geometric mean fluorescence intensity. Tfh were identified as CD4+, CD45RAneg, CXCR5+ T cells. **P=0.01-0.001, ****P<0.0001, NS=not significant, and AUC=area under the curve. FIGS. 4G-4H shows the (FIG. 4G) % CD83⁺ and (FIG. 4H) CD83 gMFI among age-associated (ABC) (CD19⁺, CD21⁻, CD11c⁺), IgD⁺ memory (CD19⁺, IgD⁺, CD38^{lo}, CD27⁺), pregerminal (CD19⁺, IgD⁺, CD38+, CD27⁺), transitional (CD19⁺, IgD⁺, CD38⁺, CD27⁻), plasmablast (CD19⁺, IgD^{lo}, CD38^{hi}, CD27⁺), and postgerminal memory (CD19⁺, IgD^{lo}, CD38^{lo}, CD27⁺) B cell subsets from a group of patients with chronic GVHD (n=9). *P<0.05, **P=0.01-0.001, ****P<0.0001, NS=not significant, and AUC=area under the curve.

FIGS. 5A-5C: Increased B cell or Tfh CD83 expression after day +100 is associated with reduced survival after alloHCT. The graphs show the overall survival of (95% CI) alloHCT recipients (n=37) based on (FIG. 5A) B cell or (FIG. 5B) T helper follicular (Tfh) cell CD83 expression. CD83 expression was determined at time of chronic GVHD diagnosis (n=17) or sampled among time-matched controls (n=20). A gMFI cutoff of 396 and 469 was used to determine low versus high CD83 expression for B cells and Tfh, respectively. FIG. 5C is a graph showing overall survival based on the diagnosis of chronic GVHD after day +100.

FIGS. 6A-6C: Auto-activated B cells from patients with chronic GvHD were stimulated with antihuman IgM and OP9-DL1 cells and cultured with anti-CD19 CAR T cells, anti-CD83 CAR T cells, or untransduced T cells for 72 hours. The graphs show the absolute number of (FIG. 6A) CD83+ B cells and (FIG. 6B) the total number of B cells recovered after the 72-hour co-culture. n=4 independent experiments. *P<0.05, **P=0.01-0.001, ***P=0.001-0.0001, and NS=not significant. The CD83 CAR T cells eliminated the autoreactive CD83 positive B cells, but did not cause complete B cell aplasia observed with CD19 CAR T cell therapy. The data show that patients at risk of chronic GvHD (i.e., identified using the methods described herein) may be treated with CD83 CAR T cells, while preserving B cell function to fight infections. This is a significant advantage to CD19 CAR T cell treatment, which can cause life-long B cell aplasia and, by extension, life-long risk for opportunistic infections and need for IV-Ig replacement therapy, which is only partially protective against opportunistic infections.

### DETAILED DESCRIPTION

The disclosure is based, at least in part, on the surprising discovery that CD83 expression in various immune cells is an indicator of the onset of allo- or autoimmunity, thereby allowing a means for identifying subjects at risk of developing an alloimmune condition or autoimmune condition or identifying subjects in the very early stages of an allo- or autoimmune condition. The materials and methods described herein allow for more informed decisions as to whether and when to treat subjects with therapeutics effective against alloimmune and/or autoimmune conditions.

In particular, the data presented herein demonstrate that CD83+ CD4+ T cells are increased among alloHCT recipients with grade II-IV acute GVHD. CD83 expression is exceedingly low on regulatory T cells or CD8+ T cells, which are critical for durable immune tolerance and beneficial graft-versus-leukemia. The data presented herein demonstrate that the frequency of peripheral CD83+ CD4+ T cells and the intensity of CD83 expression are significantly increased among patients with grade II-IV acute GvHD, and this correlates with disease onset. The materials and methods of the disclosure can inform the timing of intervention therapies (e.g., CD83 CAR T administration or other anti-CD83 targeted therapy) to prevent or treat acute GvHD. A significant survival advantage also was identified for those with low CD83 expression on circulating CD4+ T cells after alloHCT.

The disclosure also is based, at least in part, on the discovery that CD83 expression on B cells and T helper follicular cells is increased upon chronic GvHD onset. Autoreactive B cells and T helper follicular (Tfh) cells (a subset of CD4+ helper T cells) contribute to chronic GVHD pathogenesis. The frequency of CD83+ B cells and Tfh, but not monocytes, were significantly increased among patients with chronic GVHD. Further, CD83 expression on B cells and Tfh significantly correlated with chronic GVHD onset. Additionally, low CD83 expression on either B cells or Tfh was determined to be associated with a significant survival advantage after alloHCT. Thus, CD83 expression on B cell and Tfh cells, implicated in chronic GvHD, can serve as a biomarker for disease.

The disclosure provides a method of treating or reducing the risk of developing an alloimmune or autoimmune condition in a subject in need thereof. The method comprises measuring CD83 in a population of immune cells from the subject and administering to the subject a CD83-targeted therapeutic.

In various aspects, the condition is GvHD. The GvHD may be acute GvHD or may be chronic GvHD. While the symptoms of acute and chronic GvHD overlap, acute and chronic GvHD are immunologically distinct complications (e.g., complications of alloHCT). Acute GvHD is primarily mediated by alloreactive T cells. Acute GvHD is potentiated by early tissue damage during the conditioning regimens (e.g., radiation and/or chemotherapy) used to prepare patients to receive allogeneic hematopoietic cells. Further, disruption of the host gut microbiota further fuels alloreactivity by the lack of intestinal butyrate. Acute GvHD typically impacts the skin, gut, and liver and often occurs before day 100 post-transplant. Current strategies used to prevent acute GvHD primarily rely upon broad inhibition of donor T cell function, which often fails to mediate immune tolerance, impairs graft-versus leukemia, and can increase the risk for opportunistic infections. The data provided herein allows use of CD83 expression on alloreactive donor CD4+ T cells to determine those at highest risk of acute GvHD and eliminate that risk with CD83-targeted therapy.

Chronic GvHD stems from a unique triad of thymic impairment, auto- and alloantibody production by reactive B cells, and systemic tissue fibrosis. Chronic GvHD is a systemic disease affecting host mucocutaneous tissues, musculoskeletal system, gastrointestinal tract, liver, lungs, immune system, and cardiovascular system. Chronic GvHD typically occurs later after transplantation (e.g., after day 100 post alloHCT), and can essentially develop at any time, even decades after the procedure. Similar to acute GvHD, treatment of chronic GvHD requires the use of broadly immune suppressive medications. The ability to track chronic GvHD risk by the expression of CD83 on B cells and Tfh has robust clinical impact.

In various aspects of the disclosure, the measuring step is performed prior to the onset of GvHD symptoms (i.e., the subject is not suffering from one or more symptoms of GvHD or other allo- or autoimmune condition). Common symptoms of acute GvHD have been reported in the literature and include, but are not limited to, skin rash or reddening, discoloration of the skin and/or eyes (Jaundice), nausea, vomiting, diarrhea, and abdominal cramping. Similarly, common symptoms of chronic GvHD have been reported in the literature and include, but are not limited to, skin rash or discoloration, tightness or thickening of the skin, discoloration of the eyes, vision changes, dry mouth or oral ulcers, difficulty swallowing, weight loss, abdominal swelling, shortness of breath, fatigue, muscle weakness or pain, sensitivity or tightness in the joints, cytopenia, or pericarditis. GvHD is monitored using a number of techniques, including (but not limited to) clinical examination, X-ray, CT scan, liver function tests, PET scan, MRI, capsule endoscopy, and biopsy.

The method of the disclosure comprises measuring CD83 in a population of immune cells. Any suitable immune cell type may be utilized, including, e.g., CD4+ T cells, CD19+ B cells, and/or T helper follicular cells. Methods of identifying particular immune cell types are known in the art and include fluorescence-activated cell sorting (FACS).

CD83 may be measured in a variety of ways. In various aspects, the method comprises measuring CD83 protein levels. Methods of determining protein levels or identifying expression of proteins in a target cell type are known in the art. Optionally, CD83 protein levels are determined via western blot or dot blot analysis, immunohistochemistry (IHC, e.g., quantitative immunohistochemistry), flow cytometry, immunocytochemistry, enzyme-linked immunosorbent assay (ELISA), enzyme-linked immunosorbent spot (ELISPOT; Coligan, J. E., et al., eds. (1995) Current Protocols in Immunology. Wiley, New York), radioimmunoassay, or chemiluminescent immunoassay. CD83 protein levels also may be measured via geometric mean fluorescence intensity (gMFI). CD83 may be quantified and monitored in real-time on cells of interest (e.g., CD4+ T cells, Tfh, or B cells) by, for example, peripheral blood draws and measured by flow cytometry. Flow cytometry is a feasible method of tracking various cell markers of GVHD, such as phosphorylated STAT3, as early as 21 days after allogeneic hematopoietic cell transplantation. See, e.g., Pidala, J., et al., (2021) Clinical Cancer Research. 27(10):2712-2722.

Alternatively or in addition, measuring CD83 in a population of immune cells from the subject comprises measuring CD83 RNA levels. In this regard, CD83 RNA is optionally measured via real time polymerase chain reaction (qRT-PCR) or RNA sequencing. The sequence of CD83 is known in the art. See, e.g., Genbank Accession Nos. NM_001040280 and NM_004233.

CD83 protein levels may be expressed as a quantity or concentration of CD83 found in an immune cell population, or it may be expressed as a percentage of immune cells expressing CD83.

In various aspects of the disclosure, the condition is acute GvHD, and the population of immune cells comprises CD4+ T cells. In this regard, the measuring step optionally comprises detecting CD83 expression in at least 40% of CD4+ T cells in the population (e.g., at least 45%, at least 55%, or at least 60% of the cells in the population). The methodology may include (but does not require) flow cytometry, staining for live/dead cells (optionally including dump gate for CD19, CD16, CD14, etc.), CD3+, CD4+, and CD83+. As demonstrated by the data provided herein, CD83 expression in at least 40% of CD4+ T cells in a subject (e.g., a subject administered allogeneic hematopoietic cell transplantation) is associated with progression to Grade II-IV acute GvHD. Alternatively or in addition, the measuring step comprises measuring CD83 using Geometric mean fluorescence intensity (gMFI) and detecting a gMFI of at least 740.5. Also, optionally, the method is performed within 30 days (e.g., within 25 days, within 20 days, within 15 days, within 10 days, or within 5 days) of the subject receiving a transplant, such as an allogeneic hematopoietic cell transplant.

In alternative aspects of the disclosure, the condition is chronic GvHD and the population of immune cells comprises CD19+ B cells and/or T helper follicular cells. For example, the disclosure contemplates a method comprising detecting CD83 expression in at least 19% of CD19+ B cells in the population (e.g., at least 20%, at least 25%, or at least 30% of CD19+ B cells) and/or measuring CD19+ B cell CD83 using gMFI and detecting a gMFI of at least 396. The disclosure also provides a method comprising detecting CD83 expression in at least 25% of T helper follicular cells (e.g., a least 30%, at least 35%, or at least 40%) in the population and/or measuring T helper follicular cell CD83 using gMFI and detecting a gMFI of at least 469. Optionally, the method is performed after 60 days (e.g., after 75 days, after 90 days, or after 120 days) of the subject receiving a transplant (e.g., an allogeneic hematopoietic cell transplant). Optionally the method is performed prior to the onset of chronic GvHD symptoms.

While the disclosure above references GvHD, the method of the disclosure is suitable for use in the context of other autoimmune or alloimmune conditions. In various aspects, the condition is systemic lupus erythematosus (SLE), multiple sclerosis, sjogren's syndrome, systemic sclerosis/scleroderma, inflammatory bowel disease, or rheumatoid arthritis.

The term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a condition or disease of the present disclosure can provide any amount or any level of treatment. Furthermore, the treatment provided by the method may include treatment of one or more conditions or symptoms or signs of the disease being treated. For instance, the treatment method of the present disclosure may inhibit one or more symptoms of the disease. Also, the treatment provided by the methods of the present disclosure may encompass slowing the progression of the disease. The term “treat” also encompasses prophylactic treatment of the disease. Accordingly, the treatment provided by the presently disclosed method may delay the onset or reoccurrence/relapse of the disease being prophylactically treated. In exemplary aspects, the method delays the onset of the condition by 1 day, 2 days, 4 days, 6 days, 8 days, 10 days, 15 days, 30 days, two months, 4 months, 6 months, 1 year, 2 years, 4 years, or more. The prophylactic treatment encompasses reducing the risk of the condition being treated. In exemplary aspects, the method reduces the risk of the condition 2-fold, 5-fold, 10-fold, 20-fold, 50-fold, 100-fold, or more. Thus, the term “therapeutically effective” refers to a sufficient quantity of a composition which ameliorates one or more causes or symptoms of a condition or disease.

In certain aspects, the method of treating the disease may be regarded as a method of inhibiting the disease or a symptom thereof. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. The presently disclosed methods may inhibit the onset or re-occurrence of the condition or a symptom thereof to any amount or level. In exemplary embodiments, the inhibition provided by the methods is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition).

The subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). Preferably, the mammal is a human.

In various aspects of the disclosure, the method comprises administering to the subject a CD83-targeted therapeutic. A “CD83-targeted therapeutic” is a biological agent which provides a beneficial biological response with respect to treating or inhibiting the alloimmune or autoimmune condition and which specifically binds CD83 on the immune cell surface. An agent “specifically binds” to a target if it reacts or associates more frequently, more rapidly, with greater duration and/or with greater affinity with a particular target (e.g., CD83) than it does with alternative targets (e.g., other cell surface proteins). For example, a CD83-targeted therapeutic that specifically binds CD83 is one that binds CD83 with greater affinity, avidity, more readily, and/or with greater duration than it binds to other non-CD83 proteins. It is also understood that, for example, a CD83-targeted therapeutic which specifically or preferentially binds to a first target may or may not specifically or preferentially bind to a second target. As such, “specific binding” does not necessarily require (although it can include) exclusive binding. In general, under designated assay conditions, a CD83-targeted therapeutic binds preferentially to a particular target molecule and does not bind in a significant amount to other components present in a test sample.

A variety of assay formats may be used to select or characterize a CD83-targeted therapeutic. For example, solid-phase ELISA immunoassay, immunoprecipitation, BIAcore™ (GE Healthcare, Piscataway, NJ), fluorescence-activated cell sorting (FACS), Octet™ (ForteBio, Inc., Menlo Park, CA) and Western blot analysis are among many assays that may be used to characterize binding to CD83. Typically, a specific or selective reaction will be at least twice background signal or noise and more typically more than 10 times background. Even more specifically, a CD83-targeted therapeutic is said to “specifically bind” CD83 when the equilibrium dissociation constant (K_{D}) value is <1 μM, such as <100 nM, <10 nM, <100 pM, <10 pM, or <1 pM.

In various aspects, the CD83-targeted therapeutic is an antibody that binds CD83, an antigen-binding antibody fragment that binds CD83 (e.g., an antigen binding portion of an antibody), or an antibody-like construct that binds CD83. The term “antibody” refers to an intact antigen-binding immunoglobulin. The antibody can be an IgA, IgD, IgE, IgG, or IgM antibody, including any one of IgG1, IgG2, IgG3, or IgG4. In various embodiments, an intact antibody comprises two full-length heavy chains and two full-length light chains. An antibody has a variable region and a constant region. In IgG formats, a variable region is generally about 100-110 or more amino acids, comprises three complementarity determining regions (CDRs), is primarily responsible for antigen recognition, and substantially varies among other antibodies that bind to different antigens. CDR sequences are described further below. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra). The constant region allows the antibody to recruit cells and molecules of the immune system.

The architecture of antibodies has been exploited to create a growing range of alternative formats that span a molecular-weight range of at least about 12-150 kDa and have a valency (n) range from monomeric, to dimeric, to trimeric, to tetrameric, and potentially higher; such alternative formats are referred to herein as “antibody-like constructs.” Antibody-like constructs include those based on the full antibody structure and those that mimic antibody fragments which retain full antigen-binding capacity, e.g., scFvs, Fabs and VHH. The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment (fragment, antigen-binding). Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create different antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody-like construct protein product. The structure of a peptibody consists of a biologically active peptide grafted onto an Fc domain. Peptibodies are described in the art. See, e.g., Shimamoto et al., mAbs 4(5): 586-591 (2012).

Other antibody protein products include a single chain antibody (SCA), a diabody, a triabody, a tetrabody, bispecific or trispecific antibodies, and the like. Bispecific antibodies can be divided into five major classes: BsIgG, appended IgG, BsAb fragments (e.g., bispecific single chain antibodies), bispecific fusion proteins (e.g., antigen binding domains fused to an effector moiety), and BsAb conjugates. See, e.g., Spiess at al., Molecular Immunology 67(2) Part A: 97-106 (2015). Examples of bispecific antibody constructs also include, but are not limited to, tandem scFvs, bispecific T cell engager (BiTE®) format (a fusion protein consisting of two single-chain variable fragments (scFvs) joined by a linker), and Fab2 bispecifics. See, e.g., Chames & Baty, 2009, mAbs 1[6]:1-9; and Holliger & Hudson, 2005, Nature Biotechnology 23[9]:1126-1136; Wu at al., 2007, Nature Biotechnology 25[11]:1290-1297; Michaelson et al., 2009, mAbs 1[2]:128-141; International Patent Publication Nos. WO 2009032782 and WO 2006020258; Zuo et al., 2000, Protein Engineering 13[5]:361-367; U.S. Patent Application Publication No. 20020103345; Shen at al., 2006, J Biol Chem 281[16]:10706-10714; Lu et al., 2005, J Biol Chem 280[20]:19665-19672; and Kontermann, 2012 MAbs 4(2):182, all of which are expressly incorporated herein. Multispecific antibody constructs, such as trispecific antibody constructs (including three binding domains) or constructs having more than three (e.g. four, five, or more) specificities, also are contemplated.

The antibodies (or antigen-binding fragments thereof or antibody-like construct) may be a human antibody (i.e., having one or more variable and constant regions derived from human immunoglobulin sequences), humanized (i.e., have a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject), or chimeric (i.e., containing one or more regions from one antibody and one or more regions from one or more other antibodies).

In various aspects of the disclosure, the CD83-targeted therapeutic is a nanobody, i.e., an antibody-like construct based on heavy-chain only antibodies (HcAbs) in camelids. HcAbs consist of just two heavy chains with a single variable domain (VHH). In various aspects, the nanobody of the disclosure comprises a VHH domain and one or two constant domains (e.g., CH2 and CH3).

In some aspects, the antibody, antigen-binding fragment, or antibody-like construct is linked (or conjugated) to one or more moieties. For example, the conjugate moiety may comprise a therapeutic agent, a radioactive isotope, a detectable label, a pharmacokinetic modifying moiety (e.g., human serum albumin), or a purifying moiety (e.g., tag). The conjugate moiety may be covalently attached either directly to the therapeutic or via a linker.

In various aspects, the CD83-targeted therapeutic is an immune effector cell genetically modified to express a chimeric antigen receptor (CAR) polypeptide that selectively binds CD83. These cells are preferably obtained from the subject to be treated (i.e., are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

The immune effector cells may comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. Indeed, examples of suitable immune effector cells include, but are not limited to Natural Killer (NK) cells, cytotoxic T cells, regulatory T cells, and innate lymphoid cells (types 1-3).

T cells or T lymphocytes can be distinguished from other lymphocytes, such as B cells and natural killer cells (NK cells), by the presence of a T-cell receptor (TCR) on the cell surface. T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, inducing T_{H}1, T_{H}2, T_{H}3, T_{H}7, T_{H}9, or TFH, which secrete different cytokines to facilitate a different type of immune response.

Cytotoxic T cells (Tc cells or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4+ or CD8+. Memory T cells typically express the cell surface protein CD45RO.

Regulatory T cells (Treg cells), formerly known as suppressor T cells, dampen T cell-mediated immunity toward the end of an immune reaction and to suppress auto-reactive T cells that escaped the process of negative selection in the thymus. Two major classes of CD4+ Treg cells have been described-naturally occurring Treg cells and adaptive Treg cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

The T cells may comprise a mixture of CD4+ cells. In other embodiments, the T cells are enriched for one or more subsets based on cell surface expression. In some instances, the T cells are cytotoxic CD8+ T lymphocytes. In some embodiments, the T cells comprise gamma-delta T cells, which possess a distinct T-cell receptor (TCR) having one gamma chain and one delta chain instead of alpha and beta chains.

Natural-killer (NK) cells are CD56+CD3− large granular lymphocytes that kill virally infected and transformed cells and constitute a critical cellular subset of the innate immune system (Godfrey J, et al. Leuk Lymphoma 2012 53:1666-1676). Unlike cytotoxic CD8+ T lymphocytes, NK cells launch cytotoxicity without the requirement for prior sensitization, and can also eradicate MHC-I-negative cells (Nami-Mancinelli E, et al. Int Immunol 201123:427-431).

Innate lymphoid cells are tissue-resident innate lymphocytes that produce particular cytokines in response to infection, inflammation, and tissue injury. Type 1 innate lymphoid cells (ILC1s) produce interferon (IFN)-γ. Type 2 innate lymphoid cells (ILC2s) express CRTH2, KLRG1, SST2, CD161, and CD25, and produce amphiregulin, and type 2 cytokines (e.g., IL-4, IL-5, and IL-13). Type 3 innate lymphoid cells (ILC3s) produce IL-22 as well as IL-17, IL-22, IFN-γ, and GM-CSF, and can display NKp44, NKp30, and/or CD56 on the cell surface.

“Chimeric antigen receptor” or “CAR” refers to an artificial immune cell receptor that is engineered to recognize and bind to an antigen expressed by a target cell, such as a tumor cell. Generally, a CAR is designed for a T cell and is a chimera of a signaling domain of the T cell receptor (TCR) complex and an antigen-recognizing domain (e.g., a single chain fragment (scFv) of an antibody or other antibody fragment) (Enblad et al., Human Gene Therapy. 2015; 26(8):498-505). CARs have the ability to redirect immune cell specificity and reactivity toward a selected target in a non-MHC-restricted manner. The non-MHC-restricted antigen recognition gives, e.g., T cells expressing CARs the ability to recognize an antigen independent of antigen processing, thus bypassing a major mechanism of tumor escape. Moreover, when expressed in T cells, CARs advantageously do not dimerize with endogenous T-cell receptor (TCR) alpha and beta chains.

There are various formats of CARs, each of which contains different components. “First generation” CARs join an antibody-derived scFv to the CD3zeta (ζ or z) intracellular signaling domain of the immune cell receptor through hinge and transmembrane domains. “Second generation” CARs incorporate an additional domain, e.g., CD28, 4-1BB (41BB), or ICOS, to supply a costimulatory signal. “Third generation” CARs contain two costimulatory domains fused with, e.g., the TCR CD3zeta chain. Third generation costimulatory domains may include, e.g., a combination of CD3zeta, CD27, CD28, 4-1BB, ICOS, or OX40. CARs, in some embodiments, contain an ectodomain (e.g., CD3zeta), commonly derived from a single chain variable fragment (scFv), a hinge, a transmembrane domain, and an endodomain with one (first generation), two (second generation), or three (third generation) signaling domains derived from CD3 and/or co-stimulatory molecules (Maude et al., Blood. 2015; 125(26):4017-4023; Kakarla and Gottschalk, Cancer J. 2014; 20(2):151-155).

CD83-specific chimeric antigen receptors (CAR) that can be expressed in immune effector cells to suppress alloreactive donor cells have been described in, e.g., U.S. Patent Publication Nos. 20210032336 and 20200108098, each of which is incorporated herein by reference in their entireties and in particular with respect to the description of CD83 chimeric antigen receptor-bearing immune cells. In various aspects of the disclosure, the chimeric antigen receptor comprises Fab or a single-chain variable fragment (scFv) of an antibody that specifically binds CD83 or a natural ligand of CD83.

Examples of CAR polypeptides that bind CD83 include, but are not limited to, antibody-like constructs wherein the VH CDR1 sequence comprises the amino acid sequence GFSITTGGYWWT (SEQ ID NO:1), SDGIS (SEQ ID NO:7), or SNAMI (SEQ ID NO:13); the VH CDR2 sequence comprises the amino acid sequence GYIFSSGNTNYNPSIKS (SEQ ID NO:2), IISSGGNTYYASWAKG (SEQ ID NO:8), or AMDSNSRTYYATWAKG (SEQ ID NO:14); the VH CDR3 sequence comprises the amino acid sequence CARAYGKLGFDY (SEQ ID NO:3), WGGTYSI (SEQ ID NO:9), or GDGGSSDYTEM (SEQ ID NO:15); the VL CDR1 sequence comprises the amino acid sequence TLSSQHSTYTIG (SEQ ID NO:4), QSSQSVYNNDFLS (SEQ ID NO:10), or QSSQSVYGNNELS (SEQ ID NO:16); the VL CDR2 sequence comprises the amino acid sequence VNSDGSHSKGD (SEQ ID NO:5), YASTLAS (SEQ ID NO:11), or QASSLAS (SEQ ID NO:17); and the VL CDR3 sequence comprises the amino acid sequence GSSDSSGYV (SEQ ID NO:6), TGTYGNSAWYEDA (SEQ ID NO:12), or LGEYSISADNH (SEQ ID NO:18).

For example, the CAR may contain the combinations of CDR sequences provided in Table 1.

| VH CDR1 | VH CDR 2 | VH CDR3 | VL CDR1 | VL CDR2 | VL CDR3 |
|---|---|---|---|---|---|
| GFSITTGG | GYIFSSGNTNY | CARAYGK | TLSSQHST | VNSDGSH | GSSDSSGY |
| YWWT | NPSIKS (SEQ | LGFDY | YTIG (SEQ | SKGD | V (SEQ ID |
| (SEQ ID | ID NO: 2) | (SEQ ID | ID NO: 4) | (SEQ ID | NO: 6) |
| NO: 1) | | NO: 3) | | NO: 5) | |
| SDGIS | IISSGGNTYYA | WGGTYSI | QSSQSVYN | YASTLAS | TGTYGNSA |
| (SEQ ID | SWAKG (SEQ | (SEQ ID | NDFLS | (SEQ ID | WYEDA |
| NO: 7) | ID NO: 8) | NO: 9) | (SEQ ID | NO: 11) | (SEQ ID |
| | | | NO: 10) | | NO: 12) |
| SNAMI | AMDSNSRTYY | GDGGSSD | QSSQSVYG | QASSLAS | LGEYSISAD |
| (SEQ ID | ATWAKG (SEQ | YTEM | NNELS | (SEQ ID | NH (SEQ |
| NO: 13) | ID NO: 14) | (SEQ ID | (SEQ ID | NO: 17) | ID NO: 18) |
| | | NO: 15) | NO: 16) | | |

In various aspects of the disclosure, the CD83 binding portion of the CAR is an scFv.

In some embodiments, the anti-CD83 scFv VH domain comprises the amino acid sequence: (SEQ ID NO: 19, VH-GBM00)

QVQLKESGPGLVKPSQSLSLTCSVTGFSITTGGYWWTWIRQFPGQKLEWM
GYIFSSGNTNYNPSIKSRISITRDTSKNQFFLQLNSVTTEGDTARYYCAR
AYGKLGFDYWGQGTIVIVSS.

In some embodiments, the anti-CD83 scFv VL domain comprises the amino acid sequence: (SEQ ID NO: 20, VL-GBM00)

QPVLTQSPSASASLGNSVKITCTISSQHSTYTIGWYQQHPDKAPKYVMYV
NSDGSHSKGDGIPDRFSGSSSGAHRYLSIS NIQPEDEADYFCGSSDSSG
YVFGSGTQLTVL.

In some embodiments, the anti-CD83 scFv VH domain comprises the amino acid sequence: (SEQ ID NO: 21, 20D04)

METGLRWLLLVAVLKGVQCQSVEESGGRLVTPGTPLTLTCTVSGFSLSNN
AINWVRQAPGKGLEWIGYIWSGGLTYYANW AEGRFTISKTSTTVDLKMT
SPTIEDTATYFCARGINNSALWGPGTLVTVSSGQPKAPSVFPLAPCCGDT
PSSTVTLGCLVKGYLPEPVTVTWNSGTLTNGVRTFPSVRQSSGLYSLSSV
V SVTSSSQPVTCNVAHPATNTKVDKTVAPSTCSKPTCPPPELLGGPSVF
IFPPKPKDTLMISRTPEVTCVVVDVSQDDPEVQFTWYINNEQVRTARPPL
REQQFNSTIRWSTLPIAHQDWL RGKEFKCKVHNKALPAPIEKTISKARG
QPLEPKVYTMGPPREELSSRSVSLTCMINGFYPSDISVEWEKNGKAEDNY
KTTPAVLDSDGSYFLYNKLSVPTSEWQRGDVFTCSVMHEALHN HYTQKS
ISRSPGK.

In some embodiments, the anti-CD83 scFv VL domain comprises the amino acid sequence: (SEQ ID NO: 22, 20D04)

MDMRAPTQLLGLLLLWLPGARCADVVMTQTPASVSAAVGGTVTINCQASE
SISNYLSWYQQKPGQPPKLLIYRTSTLASG VSSRFKGSGSGTEYTLTIS
GVQCDDVATYYCQCTSGGKFISDGAAFGGGTEWVKGDPVAPTVLLFPPSS
DEVATGTVTIVCVANKYFPDVTVTWEVDGTTQTTGIENSKTPQNSADCTY
N LSSTLTLTSTQYNSHKEYTCKVTQGTTSVVQSFSRKNC.

In some embodiments, the anti-CD83 scFv VH domain comprises the amino acid sequence: (SEQ ID NO: 23, 11G05)

METGLRWLLLVAVLKGVQCQSVEESGGRLVTPGTPLTLTCTVSGFTISDY
DLSWVRQAPGEGLKYIGFIAIDGNPYYATW AKGRFTISKTSTTVDLKIT
APTTEDTATYFCARGAGDLWGPGTLVTVSSGQPKAPSVFPLAPCCGDTPS
STVTLGCLVKGYLPEPVTVTWNSGTLTNGVRTFPSVRQSSGLYSLSSVVS
V TSSSQPVTCNVAHPATNTKVDKTVAPSTCSKPTCPPPELLGGPSVFIF
PPKPKDTLMISRTPEVTCVVVDVSQDDPEVQFTWYINNEQVRTARPPLRE
QQFNSTIRVVSTLPIAHQDWLR GKEFKCKVHNKALPAPIEKTISKARGQ
PLEPKVYTMGPPREELSSRSVSLTCMINGFYPSDISVEWEKNGKAEDNYK
TTPAVLDSDGSYFLYNKLSVPTSEWQRGDVFTCSVMHEALHNH YTQKSI
SRSPGK.

In some embodiments, the anti-CD83 scFv VL domain comprises the amino acid sequence: (SEQ ID NO: 24, 11G05)

MDTREPTQLLGLLLLWLPGARCADVVMTQTPASVSAAVGGTVTINCQSSK
NVYNNNWLSWFQQKPGQPPKLLIYYASTLA SGVPSRFRGSGSGTQFTLT
ISDVQCDDAATYYCAGDYSSSSDNGFGGGTEVVVKGDPVAPTVLLFPPSS
DEVATGTVTIVCVANKYFPDVTVTWEVDGTTQTTGIENSKTPQNSADCTY
N LSSTLTLTSTQYNSHKEYTCKVTQGTTSVVQSFSRKNC.

In some embodiments, the anti-CD83 scFv VH domain comprises the amino acid sequence: (SEQ ID NO: 25, 14C12)

METGLRWLLLVAVLKGVHCQSVEESGGRLVTPGTPLTLTCTASGFSRSSY
DMSWVRQAPGKGLEWVGVISTAYNSHYASWAKGRFTISRTSTTVDLKMTS
LTTEDTATYFCARGGSWLDLWGQGTLVTVSSGQPKAPSVFPLAPCCGDTP
SSTVTLGCLVKGYLPEPVTVTWNSGTLTNGVRTFPSVRQSSGLYSLSSVV
SVTSSSQPVTCNVAHPATNTKVDKTVAPSTCSKPTCPPPELLGGPSVFIF
PPKPKDTLMISRTPEVTCVWDVSQDDPEVQFTWYINNEQVRTARPPLREQ
QFNSTIRVVSTLPIAHQDWL RGKEFKCKVHNKALPAPIEKTISKARGQP
LEPKVYTMGPPREELSSRSVSLTCMINGFYPSDISVEWEKNGKAEDNYKT
TPAVLDSDGSYFLYNKLSVPTSEWQRGDVFTCSVMHEALHN HYTQKSIS
RSPGK.

In some embodiments, the anti-CD83 scFv VL domain comprises the amino acid sequence: (SEQ ID NO: 26, 14C12)

MDXRAPTQLLGLLLLWLPGARCALVMTQTPASVSAAVGGTVTINCQSSQ
SVYDNDELSWYQQKPGQPPKLLIYALASKLASGVPSRFKGSGSGTQFAL
TISGVQCDDAATYYCQATHYSSDWYLTFGGGTEVVVKGFPVAPTVLLFP
PSSDEVATGTVTIVCVANKYFPDVTVTWEVDGTTQTTGTENSKTPQNSA
DCTYNLSSTLTLTSTQYNSHKEYTCKVTQGTTSVVQSFSRKNC.

In some embodiments, the anti-CD83 scFv VH domain comprises the amino acid sequence: (SEQ ID NO: 27, 020B08)

METGLRWLLLVAVLKGVQCQSVEESGGRLVTPGTPLTLCTVSGFSLSSY
DMTWVRQAPGKGLEWIGIIYASGTTYYANWAKGRFTISKTSTTVDLKVT
SPTIGDTATYFCAREGAGVSMTLWGPGTLVTVSSGQPKAPSVFPLAPCC
GDTPSSTVTLGCLVKGYLPEPVTVTWNSGTLTNGVRTFPSVRQSSGLYS
LSSVVSVTSSSQPVTCNVAHPATNTKVDKTVAPSTCSKPTCPPPELLGG
PSVFIFPPKPKDTLMISRTPEVTCVVVDVSQDDPEVQFTWYINNEQVRT
ARPPLREQQFNSTIRVVSTLPIAHQDWLRGKEFKCKVHNKALPAPIEKT
ISKARGQPLEPKVYTMGPPREELSSRSVSLTCMINGFYPSDISVEWEKN
GKAEDNYKTTPAVLDSDGSYFLYNKLSVPTSEWQRGDVFTCSVMHEALH
NHYTQKSISRSPGK.

In some embodiments, the anti-CD83 scFv VL domain comprises the amino acid sequence: (SEQ ID NO: 28, 020B08)

MDMRAPTQLLGLLLLWLPGARCAYDMTQTPASVEVAVGGTVTIKCQASQ
SISTYLDWYQQKPGQPPKLLIYDASDLASGVPSRFKGSGSGTQFTLTIS
DLECADAATYYCQQGYTHSNVDNVFGGGTEVVVKGDPVAPTVLLFPPSS
DEVATGTVTIVCVANKYFPDVTVTWEVDGTTQTTGIENSKTPQNSADCT
YNLSSTLTLTSTQYNSHKEYTCKVTQGTTSVVQSFSRKNC.

In some embodiments, the anti-CD83 scFv VH domain comprises the amino acid sequence: (SEQ ID NO: 29, 006G05)

METGLRWLLLVAVLKGVQCQSVEESGGRLVSPGTPLTLTCTASGFSLSSY
DMSWVRQAPGKGLEYIGIISSSGSTYYASWAKGRFTISKTSTTVDLEVTS
LTTEDTATYFCSREHAGYSGDTGHLWGPGTLVTVSSGQPKAPSVFPLAPC
CGDTPSSTVTLGCLVKGYLPEPVTVTWNSGTLTNGVRTFPSVRQSSGLYS
ELLGGPSVGIGPPKPKDTLMISRTPEVTCVVVDVSQDDPEVQFTWYINNE
QLSSVVSVTSSSQPVTCNVAHPATNTKVDKTVAPSTCSKPTCPPPVRTAR
PPLREQQFNSTIRVVSTLPIAHQDWLRGKEFKCKVHNKALPAPIEKTISK
ARGQPLEPKVYTMGPPREELSSRSVSLTCMINGFYPSDISVEWEKNGKAE
DNYKTTPAVLDSDGSYFLYNKLSVPTSEWQRGDVFTCSVMHEALHNHYTQ
KSISRSPGK.

In some embodiments, the anti-CD83 scFv VL domain comprises the amino acid sequence: (SEQ ID NO: 30, 006G05)

MDMRAPTQLLGLLLLWLPGARCAYDMTQTPASVEVAVGGTVAIKCQASQS
VSSYLAWYQQKPGQPPKPLIYEASMLAAGVSSRFKGSGSGTDFTLTISDL
ECDDAATYYCQQGYSISDIDNAFGGGTEVVVKGDPVAPTVLLFPPSSDEV
ATGTVTIVCVANKYFPDVTVTWEVDGTTQTTGIENSKTPQNSADCTYNLS
STLTLTSTQYNSHKEYTCKVTQGTTSVVQSFSRKNC.

In some embodiments, the anti-CD83 scFv VH domain comprises the amino acid sequence: (SEQ ID NO: 31, 96G08)

METGLRWLLLVAVLKGVQCQSVEESGGRLVTPGTPLTLCTVSGIDLSSDG
ISWVRQAPGKGLEWIGIISSGGNTYYASWAKGRFTISRTSTTVDLKMTSL
TTEDTATYFCARVVGGTYSIWGQGTLVTVSSASTKGPSVYPLAPGSAAQT
NSMVTLGCLVKGYFPEPVTVTWNSGSLSSGVHTFPAVLQSDLYTLSSSVT
VPSSTWPSETVTCNVAHPASSTKVDKKIVPRDCGCKPCICTVPEVSSVFI
FPPKPDVLTITLTPKVTCVVVDISKDDPEVQFSWFVDDVEVHTAQTQPRE
EQFNSTFRSVSELPIMHQDWLNGKEFKCRVNSAAFPAPIEKTISKTKGRP
KAPQVYTIPPPKEQMAKDKVSLTCMITDFFPEDITVEWQWNGQPAENYKN
TQPIMDTDGSYFVYSKLNVQKSNWEAGNTFTCSVLHEGLHNHHTEKSLSH
SPGK.

In some embodiments, the anti-CD83 scFv VL domain comprises the amino acid sequence: (SEQ ID NO: 32, 96G08)

MDTRAPTQLLGLLLLWLPGATFAQVLTQTASPVSAPVGGTVTINCQSSQS
VYNNDFLSWYQQKPGQPPKLLIYYASTLASGVPSRFKGSGSGTQFTLTIS
DLECDDAATYYCTGTYGNSAWYEDAFGGGTEVVVKRTPVAPTVLLFPPSS
AELATGTATIVCVANKYFPDGTVTWKVDGITQSSGINNSRTPQNSADCTY
NLSSTLTLSSDEYNSHDEYTCQVAQDSGSPVVQSFSRKSC.

In some embodiments, the anti-CD83 scFv VH domain comprises the amino acid sequence: (SEQ ID NO: 33, 95F04)

METGLRWLLLVAVLKGVQCQSVEESGGRLVTPGTPLTLTCTVSGIDLSSN
AMIWVRQAPREGLEWIGAMDSNSRTYYATWAKGRFTISRTSSITVDLKIT
SPTTEDTATYFCARGDGGSSDYTEMWGPGTLVTVSSASTKGPSVYPLAPG
SAAQTNSMVTLGCLVKGYFPEPVTVTWNSGSLSSGVHTFPAVLQSDLYIL
SSSVTVPSSTWPSETVTCNVAHPASSTKVDKKIVPRDCGCKPCICTVPEV
SSVFIFFPKPKDVLTITLTPKVTCVVVDISKDDPEVQFSWFVDDVEVHTA
QTQPREEQFNSTFRSVSELPIMHQDWLNGKEFKCIRVNSAAFPAPIEKTI
SKTKGRPKAPQVYTIPPPKEQMAKDKVSLTCMITDFFPEDITVEWQWNGQ
PAENYKNTQPIMDTDGSYFVYSKLNVQKSNWEAGNTFTCSVLHEGLHNHH
TEKSLSHSPGK.

In some embodiments, the anti-CD83 scFv VL domain comprises the amino acid sequence: (SEQ ID NO: 34, 95F04)

MDTRAPTQLLGLLLLWLPGATFAQAVVTQTTSPVSAPVGGTVTINCQSSQ
SVYGNNELSWYQQKPGQPPKLLIYQASSLASGVPSRFKGSGSGTQFTLTI
SDLECDDAATYYCLGEYSISADNHFGGGTEVVVKRTPVAPTVLLFPPSSA
ELATGTATIVCVANKYFPDGTVTWKVDGITQSSGINNSRTPQNSADCTYN
LSSTLTLSDEYNHDEYTCQVAQDSGSPVVQSFSRKSC.

In some embodiments, the anti-CD83 scFv VH domain comprises the amino acid sequence: (SEQ ID NO: 35)

QVQLVQSGGAVVQPGRSLRLSCAASGFTFSTYGMHWVRQAPGKGLEWVAA
VSYDGSNKYYADFVKGRFTISRDNPKNTLYLQMNSLRADDTAVYYCARRG
GLDIWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCAAA.

In some embodiments, the anti-CD83 scFv VL domain comprises the amino acid sequence: (SEQ ID NO: 36)

LTQPPPASGTPGQQRVTISCSGSSSNIGSNTVNWYQQLPGTAPKLLIYYG
NDQRPSGVPDRFSASKSGTSASLAISGLQSEDEAHYYCAAWDGSLNGGVI
FGGGTKVTLG.

In some embodiments, the anti-CD83 scFv VL domain comprises the amino acid sequence: (SEQ ID NO: 37)

VTQPPSASGTPGQRVTISCSGSSSNIGTNPVNWYQQLPGTAPKLLIYTTDQ
RPSGVPDRFSGSKSGTSASLAISGLQSEDEADYYCAAWDDSLSGLYVFGTG
TKVTVLG.

In some embodiments, the anti-CD83 scFv VL domain comprises the amino acid sequence: (SEQ ID NO: 38)

MTHTPLSLSVTPGQPASISCKSSQSLLHSDGKTYLYWYLQRPGQSPQPLIY
EVSNRFSGVPDRFSGSGSGTDFTLKISRVQAEDVGVYYCMQSLQLWFTGQG
TKVEIKR.

In some embodiments, the anti-CD83 scFv VL domain comprises the amino acid sequence: (SEQ ID NO: 39)

MTQSPLSLPVTLGQPASISCRSSGSLIHSDGNTYLDWFQQRPGQSPRRLIY
KVSNRDSGVPDRFSGSGSGTDFTLRISRVEAEDIGVYYCMQATHWPRTFGQ
GTKVEIKR.

In some embodiments, the anti-CD83 scFv VL domain comprises the amino acid sequence: (SEQ ID NO: 40)

MTQSPLSLPVTLGQPASISCRSSQSLVDSAGNTFLHWFHQRPGQSPRRLIY
KVSNRDSGVPDRFSGSGSGTDFTLKISRVEAEDVGVYYCMQGTHWPRTFGQ
GTKVEIKR.

In some embodiments, the anti-CD83 scFv VL domain comprises the amino acid sequence: (SEQ ID NO: 41)

LTQSPLSLPVTLGQPASISCKSSQSLVDSDGNTYLNWFQQRPGQSPRRL
IYKVSNRDSGVPDRFSGSGSGTDFTLKISRVEAEDVGVYYCMQGTHWPR
TFGQ GTKVEIKR.

In some embodiments, the anti-CD83 scFv VL domain comprises the amino add sequence: (SEQ ID NO: 42)

MTQSPLSLPVTLGQPASISCRSSQSLVHSDGNMYLNWFQQRPGQSPRRL
IYKVSNRDSGVPDRFSGSGSGTDFTLKISRVEAEDVGVYYCMQATQPTW
TFGQ GTKLEIKR.

In some embodiments, the anti-CD83 scFv VL domain comprises the amino acid sequence: (SEQ ID NO: 43)

MTQSPSSLSASVGDRVTITCQASQDISNYLNWYQQKPGKAPKLLIYDAS
NLETGVPSRFSGSGSGTDFTFTISSATYYCQQTYQGTKLEIKR.

In some embodiments, the anti-CD83 scFv VL domain comprises the amino acid sequence: SEQ ID NO: 44)

MTQSPSSLSASVGHPVTITCRASQSLISYLNWYHQKPGKAFKLLIYAAS
ILQSGVPSRFSGSGSGTDFTLTISSLQPENFASYYCQHTDSFPRTFGHG
TKVE IKR.

In some embodiments, the anti-CD83 scFv VL domain comprises the amino acid sequence: (SEQ ID NO: 45)

LTQPPSASGTPGQGVTISCRGSTSNIGNNVVNWYQHVPGSAPKLLIWSN
IQRPSGIPDRFSGSKSGTSASLAISGLQSEDQAVYYCAVWDDGLAGWVF
GGGT TVTVLS.

In some embodiments, the anti-CD83 scFv VL domain comprises the amino acid sequence: (SEQ ID NO: 46)

MTQAPVVSVALEQTVRITCQGDSLAIYYDFWYQHKPGQAPVLVIYGKNN
RPSGIPHRFSGSSSNTDSLTITGAQAEDEADYYCNSRDSSGNHWVFGGG
TNLT VLG.

In some embodiments, the anti-CD83 scFv VL domain comprises the amino acid sequence: (SEQ ID NO: 47)

LTQSPLSLPVTLGQPASISCKSNQSLVHSDGNTYLNWFQQRPGQSPRRL
IYKVSNRDSGVPDRFSGSGSGTDFTLKINRVEAEDVGVYYCMQGTQWPR
TFGG QGTKLDIKR.

In some embodiments, the anti-CD83 scFv VH domain has been humanized and comprises the amino acid sequence: (SEQ ID NO: 48, VH-GBM01)

QVQLQESGPGLVKPSETLSLTCTVSGFSITTGGYWWTWIRQPPGKGLEW
IGYIFSSGNTNYNPSIKSRVTISVDTSKNQFSLKLSSVTAADTAVYYCA
RAYG KLGFDYWGQGTLVTVSS.

In some embodiments, the anti-CD83 scFv VH domain has been humanized and comprises the amino acid sequence: (SEQ ID NO: 49, VH-GBM02)

QVQLQESGPGLVKPSQTLSLTCTVSGFSITTGGYWWTWIRQHPGKGLEW
IGYIFSSGNTNYNPSIKSLVTISVDTSKNQFSLKLSSVTAADTAVYYCA
RAYG KLGFDYWGQGTLVTVSS.

In some embodiments, the anti-CD83 scFv VH domain has been humanize and comprises the amino acid sequence: (SEQ ID NO: 50, VH-GBM03)

QVQLQESGPGLVKPSQTLSLTCTVSGFSITTGGYWWTWIRQPPGKGLEW
IGYIFSSGNTNYNPSIKSRVTISVDTSKNQFSLKLSSVTAADTAVYYCA
RAYG KLGFDYWGQGTLVTVSS.

In some embodiments, the anti-CD83 scFv VH domain has been humanized and comprises the amino acid sequence: (SEQ ID NO: 51, VH-GBM04)

QVQLQESGPGLVKPSETLSLTCTVSGFSITTGGYWWTWIRQPPGKGLEW
IGYIFSSGNTNYNPSIKSRVTISRDTSKNQFSLKLSSVTAADTAVYYCA
RAYG KLGFDYWGQGTLVTVSS.

In some embodiments, the anti-CD83 scFv VH domain has been humanized and comprises the amino acid sequence: (SEQ ID NO: 52, VH-GBM05)

QVQLQESGPGLVKPSETLSLTCTVSGFSITTGGYWWTWIRQPPGKGLEW
IGYIFSSGNTNYNPSIKSRVTISVDTSKNQFSLKLSSVTAADTARYYCA
RAYG KLGFDYWGQGTLVTVSS.

In some embodiments, the anti-CD83 scFv VH domain has been humanized and comprises the amino acid sequence: (SEQ ID NO: 53, VH-GBM0)

QVQLQESGPGLVKPSETLSLTCTVSGFSITTGGYWWTWIRQPPGKGLEW
IGYIFSSGNTNYNPSIKSRISITRDTSKNQFFLQLNSVTTEGDTARYYC
ARAY GKLGFDYWGQGTLVTVSS.

In some embodiments, the anti-CD83 scFv VL domain has been humanized and comprises the amino acid sequence: (SEQ ID NO: 54, VL-GBM01)

QLVLTQSPSASASLGASVKLTCTLSSQHSTYTIGWHQQQPEKGPRYLMK
VNSDGSHSKGDGIPDRFSGSSSGAERYLTISSLQSEDEADYYCGSSDSS
GYVF GSGTKVTVL.

In some embodiments, the anti-CD83 scFv VL domain has been humanized and comprises the amino acid sequence: (SEQ ID NO: 55, VL-GBM02)

LPVLTQPPSASALLGASIKLTCTLSSQHSTYTIGWYQQRPGRSPQYIMK
VNSDGSHSKGDGIPDRFMGSSSGADRYLTFSNLQSDDEAEYHCGSSDSS
GYVF GSGTKVTVL.

The heavy and light chains are optionally separated by a linker. Suitable linkers for scFv antibodies are known in the art. In some embodiments, the linker comprises the amino acid sequence GGGGSGGGGSGGGGS (SEQ ID NO:56).

In some embodiments, the anti-CD83 scFv comprises an amino acid sequence: (SEQ ID NO: 57)

QPVLTQSPSASASLGNSVKITCTLSSQHSTYTIGWYQQHPDKAPKYVMY
VNSDGSHSKGDGIPDRFSGSSSGAHRYLSISNIQPEDEADYFCGSSDSS
GYVFGSGTQLTVLRAAASSGGGGSGGGGSGGGGSQPVLTQSPSASASLG
NSVKITCTLSSQHSTYTIGWYQQHPDKAPKYVMYVNSDGSHSKGDGIPD
RFSGSSSGAHRYLSISNIQPEDEADYFCGSSDSSGYVFGSGTQLTVLRA
AA.

In some embodiments, the anti-CD83 scFv comprises an amino acid sequence: (SEQ ID NO: 58)

QVQLKESGPGLVKPSQSLSLTCSVTGFSITTGGYWWTWIRQFPGQKLEW
MGYIFSSGNTNYNPSIKSRISITRDTSKNQFFLQLNSVTTEGDTARYYC
ARAYGKLGFDYWGQGTLVTVSSGGGGSGGGGSGGGGSQVQLKESGPGIN
KPSQSLSLTCSVTGFSITTGGYWWTWIRQFPGQKLEWMGYIFSSGNTNY
NPSIKSRISITRDTSKNQFFLQLNSVTTEGDTARYYCARAYGKLGFDYW
GQGTL VTV.

In some embodiments, the anti-CD83 scFv comprises an amino acid sequence: (SEQ ID NO: 59)

QVQLQESGPGLVKPSETLSLTCTVSGFSITTGGYWWTWIRQPPGKGLEW
IGYIFSSGNTNYNPSIKSRVTISVDTSKNQFSLKLSSVTAADTAVYYCA
RAYGKLGFDYWGQGTLVTVSSGGGGSGGGGSGGGGSQLVLTQSPSASAS
LGASVKLTCTLSSQHSTYTIGWHQQQPEKGPRYLMKVNSDGSHSKGDGI
PDRFSGSSSGAERYLTISSLQSEDEADYYCGSSDSSGYVFGSGTKVTVL.

In some embodiments, the anti-CD83 scFv comprises an amino acid sequence: (SEQ ID NO: 60)

QVQLQESGPGINKPSQTLSLTCTVSGFSITTGGYWWTWIRQHPGKGLEW
IGYIFSSGNTNYNPSIKSLVTISVDTSKNQFSLKLSSVTAADTAVYYCA
RAYGKLGFDYWGQGTLVTVSSGGGGSGGGGSGGGGSQLVLTQSPSASAS
LGASVKLTCTLSSQHSTYTIGWHQQQPEKGPRYLMKVNSDGSHSKGDGI
PDRFSGSSSGAERYLTISSLQSEDEADYYCGSSDSSGYVFGSGTKVTVL.

In some embodiments, the anti-CD83 scFv comprises an amino acid sequence: (SEQ ID NO: 61)

QVQLQESGPGLVKPSQTLSLTCTVSGFSITTGGYWWTWIRQPPGKGLEW
IGYIFSSGNTNYNPSIKSRVTISVDTSKNQFSLKLSSVTAADTAVYYCA
RAYGKLGFDYWGQGTLVTVSSGGGGSGGGGSGGGGSQLVLTQSPSASAS
LGASVKLTCTLSSQHSTYTIGWHQQQPEKGPRYLMKVNSDGSHSKGDGI
PDRFSGSSSGAERYLTISSLQSEDEADYYCGSSDSSGYVFGSGTKVTVL.

In some embodiments, the anti-CD83 scFv comprises an amino acid sequence: (SEQ ID NO: 62)

QVQLQESGPGINKPSETLSLTCTVSGFSITTGGYWWTWIRQPPGKGLEW
IGYIFSSGNTNYNPSIKSRVTISRDTSKNQFSLKLSSVTAADTAVYYCA
RAYGKLGFDYWGQGTLVTVSSGGGGSGGGGSGGGGSQLVLTQSPSASAS
LGASVKLTCTLSSQHSTYTIGWHQQQPEKGPRYLMKVNSDGSHSKGDGI
PDRFSGSSSGAERYLTISSLOSEDEADYYCGSSDSSGYVFGSGTKVTVL.

In some embodiments, the anti-CD83 scFv comprises an amino acid sequence: (SEQ ID NO: 63)

QVQLQESGPGLVKPSETLSLTCTVSGFSITTGGYWWTWIRQPPGKGLEW
IGYIFSSGNTNYNPSIKSRVTISVDTSKNQFSLKLSSVTAADTARYYCA
RAYGKLGFDYWGQGTLVTVSSGGGGSGGGGSGGGGSQLVLTQSPSASAS
LGASVKLICTLSSQHSTYTIGWHQQQPEKGPRYLMKVNSDGSHSKGDGI
PDRFSGSSSGAERYLTISSLQSEDEADYYCGSSDSSGYVFGSGTKVTVL.

In some embodiments, the anti-CD83 scFv comprises an amino acid sequence: (SEQ ID NO: 64)

QVQLQESGPGLVKPSETLSLTCTVSGFSITTGGYWWTWIRQPPGKGLEW
IGYIFSSGNTNYNPSIKSRISITRDTSKNQFFLQLNSVTTEGDTARYYC
ARAYGKLGFDYWGQGTLVTVSSGGGGSGGGGSGGGGSQLVLTQSPSASA
SLGASVKLTCTLSSQHSTYTIGWHQQQPEKGPRYLMKVNSDGSHSKGDG
IPDRFSGSSSGAERYLTISSLQSEDEADYYCGSSDSSGYVFGSGTKVTV
L.

In some embodiments, the anti-CD83 scFv comprises an amino acid sequence: (SEQ ID NO: 65)

QVQLQESGPGLVKPSETLSLTCTVSGFSITTGGYWWTWIRQPPGKGLEW
IGYIFSSGNTNYNPSIKSRVTISVDTSKNQFSLKLSSVTAADTAVYYCA
RAYGKLGFDYWGQGTLVTVSSGGGGSGGGGSGGGGSLPVLTQFPSASAL
LGASIKLTCTLSSQHSTYTIGWYQQRPGRSPQYIMKVNSDGSHSKGDGI
PDRFMGSSSGADRYLTFSNLQSDDEAEYHCGSSDSSGYVFGSGTKVTVL.

In some embodiments, the anti-CD83 scFv comprises an amino acid sequence: (SEQ ID NO: 66)

QVQLQESGPGLVKPSQTLSLTCTVSGFSITTGGYWWTWIRQHPGKGLEW
IGYIFSSGNTNYNPSIKSLVTISVDTSKNQFSLKLSSVTAADTAVYYCA
RAYGKLGFDYWGQGTLVTVSSGGGGSGGGGGGGGSLPVLTQPPSASALL
GASIKLTCTLSSQHSTYTIGWYQQRPGRSPQYIMKVNSDGSHSKGDGIP
DRFMGSSSGADRYLIFSNLQSDDEAEYHCGSSDSSGYVFGSGTKVTVL.

In some embodiments, the anti-CD83 scFv comprises an amino acid sequence: (SEQ ID NO: 67)

QVQLQESGPGLVKPSQTLSLTCTVSGFSITTGGYWWTWIRQPPGKGLEW
IGYIFSSGNTNYNPSIKSRVTISVDTSKNQFSLKLSSVTAADTAVYYCA
RAYGKLGFDYWGQGTLVTVSSGGGGSGGGGSGGGGSLPVLTQPPSASAL
LGASIKLTCTLSSQHSTYTIGWYQQRPGRSPQYIMKVNSDGSHSKGDGI
PDRFMGSSSGADRYLTFSNLQSDDEAEYHCGSSDSSGYVFGSGTKVTVL.

In some embodiments, the anti-CD83 scFv comprises an amino acid sequence: (SEQ ID NO: 68)

QVQLQESGPGLVKPSETLSLTCTVSGFSITTGGYVWVTWIRQPPGKGLE
WIGYIFSSGNTNYNPSIKSRVTISRDTSKNQFSLKLSSVTAADTAVYYC
ARAYGKLGFDYWGQGTLVTVSSGGGGSGGGGSGGGGSLPVLTQFPSASA
LLGASIKLTCTLSSQHSTYTIGWYQQRPGRSPQYIMKVNSDGSHSKGDG
IPDRFMGSSSGADRYLTFSNLQSDDEAEYHCGSSDSSGYVFGSGTKVTV
L.

In some embodiments, the anti-CD83 scFv comprises an amino acid sequence: (SEQ ID NO: 69)

QVQLQESGPGLVKPSETLSLTCTVSGFSITTGGYWWTWIRQPPGKGLEW
IGYIFSSGNTNYNPSIKSRVTISVDTSKNQFSLKLSSVTAADTARYYCA
RAYGKLGFDYWGQGTLVTVSSGGGGSGGGGSGGGGSLPVLTQPPSASAL
LGASIKLTCTLSSQHSTYTIGWYQQRPGRSPQYIMKVNSDGSHSKGDGI
PDRFMGSSSGADRYLTFSNLQSDDEAEYHCGSSDSSGYVFGSGTKVTVL.

In some embodiments, the anti-CD83 scFv comprises an amino acid sequence: (SEQ ID NO: 70)

QVQLQESGPGLVKPSETLSLTCTVSGFSITTGGYWWTWIRQPPGKGLEW
IGYIFSSGNTNYNPSIKSRISITRDTSKNQFFLQLNSVTTEGDTARYYC
ARAYGKLGFDYWGQGTLVTVSSGGGGSGGGGSGGGGSLPVLTQPPSASA
LLGASIKLTCTLSSQHSTYTIGWYQQRPGRSPQYIMKVNSDGSHSKGDG
IPDRFMGSSSGADRYLTFSNLQSDDEAEYHCGSSDSSGYVFGSGTKVTV
L.

In some embodiments, the anti-CD83 scFv comprises an amino acid sequence: (SEQ ID NO: 71)

QVQLKESGPGLVKPSQSLSLTCSVTGFSITTGGYWWTWIRQFPGQKLEW
MGYIFSSGNTNYNPSIKSRISITRDTSKNQFFLQLNSVTTEGDTARYYC
ARAYGKLGFDYWGQGTLVTVSSGGGGSGGGGSGGGGSQPVLTQSPSASA
SLGNSVKITCTISSQHSTYTIGWYQQHPDKAPKYVMYVNSDGSHSKGDG
IPDRFSGSSSGAHRYLSISNIQPEDEADYFCGSSDSSGYVFGSGTQLTV
L.

It will be appreciated that the CDR sequences and VH and VL sequences disclosed herein with respect to CARs also may be utilized in the context of an antibody, antigen-binding fragment thereof, or antibody-like construct, any of which may be conjugated to an additional moiety (e.g., an antibody-drug conjugate).

The CD83-targeted therapeutic of the present disclosure may be administered by any acceptable route, including parenteral and subcutaneous. Other routes include intravenous, intradermal, intramuscular, intraperitoneal, intranodal and intrasplenic, for example. In exemplary aspects, the therapeutic is provided via systemic (e.g., intravenous) administration. A composition comprising the therapeutic typically is sterile. In certain embodiments, parenteral compositions generally are placed into a container having a sterile access port, for example, an intravenous solution bag, or vial having a stopper pierceable by a hypodermic injection needle, or a prefilled syringe.

In some embodiments, the method described herein further comprises administration of one or more other therapeutic agents. In this regard, the method optionally comprises administering to the subject a second therapeutic agent selected from the group consisting of a corticosteroid, methotrexate, cyclosporine, mycophenolate mofetil, tacrolimus, sirolimus, everolimus, antithymocyte globulin, alemtuzumab, cyclophosphamide, ibrutinib, imatinib, infliximab, etanercept, tocilizumab, alemtuzumab, basiliximab, daclizumab, rituximab, denileukin diftitox, pentostatin, ruxolitinib, belumosudil, abatacept, cyclosporine, thalidomide, halofuginone, hydroxychloroquine, mesenchymal stem cells, type 2 innate lymphoid cells, and regulatory T cells (or any combination thereof, potentially in further combination with other therapeutics).

The disclosure further provides a method of identifying a subject at risk of developing acute GvHD, the method comprising measuring CD83 in a population of CD4+ T cells from the subject, wherein the presence of CD83 expression in at least 40% of CD4+ T cells in the population or a gMFI of at least 740.5 in the population of CD4+ T cells indicates that the subject is at risk of developing acute GvHD. Also provided is a method of identifying a subject at risk of developing chronic GvHD, the method comprising measuring CD83 in a population of CD19+ B cells from the subject, wherein the presence of CD83 expression in at least 19% of CD19+ B cells in the population or a gMFI of at least 396 indicates that the subject is at risk of developing chronic GvHD. Alternatively or in addition, the method of identifying a subject at risk of developing chronic GvHD comprises measuring CD83 in a population of T helper follicular cells from the subject, wherein the presence of CD83 expression in at least 25% of T helper follicular cells in the population or a gMFI of at least 469 indicates that the subject is at risk of developing chronic GvHD.

The disclosure further provides a CD83-targeted therapeutic for use in a method of treating an alloimmune or autoimmune condition in a subject, wherein the method comprises measuring CD83 in a population of immune cells from the subject, and administering to the subject the CD83-targeted therapeutic. The disclosure further provides use of a CD83-targeted therapeutic for treating (or reducing the risk of developing or delaying the onset of) an alloimmune or autoimmune condition in a subject, wherein (a) CD83 expression is detected in at least 40% of CD4+ T cells in a population of immune cells from the subject or a CD83 gMFI of at least 740.5 is detected in the population of CD4+ T cells from the subject, (b) CD83 expression is detected in at least 19% of CD19+ B cells in a population of immune cells from the subject or a CD83 gMFI of at least 396 is detected in the population of CD19+ B cells from the subject, or (c) CD83 expression is detected in at least 25% of T helper follicular cells in the immune cell population from the subject or a CD83 gMFI of at least 469 is detected in the population of T helper follicular cells obtained from the subject. In various aspects, the subject has previously received allogeneic hematopoietic cell therapy.

### EXAMPLES

### Example 1—CD83 Expression on CD4+ T Cells is Increased Upon Acute GvHD Onset

Acute GVHD remains a major cause of morbidity and death after allogeneic hematopoietic cell transplantation (alloHCT) for blood cancers and marrow failure syndromes. Acute GVHD is typically diagnosed before day +100 and can affect recipient skin, gastrointestinal tract, and/or the liver. Current prevention strategies, like calcineurin-inhibitors, broadly suppress pathogenic donor T cells that mediate GVHD, but also impair healthy effector T cells and regulatory T cells (Treg) that mediate beneficial graft-versus-leukemia (GVL) and immune tolerance, respectively. As such, standard GVHD prevention offers incomplete protection at best and places alloHCT recipients at risk of disease relapse and opportunistic infections.

Methods: To measure CD83 expression, peripheral blood (˜50-80 mls) was serially drawn at day +21 and then weekly up to day +100, or at time of GVHD symptom onset. Peripheral blood mononuclear cells were purified by density gradient and stained for extracellular CD83 expression by flow cytometry. Using a cutoff for CD83 gMFI on CD4+ T cells of at least 740.5, “high” CD83 expression was identified on the target cell. This threshold can be used as a prognostic indicator for acute GVHD as well as a cut off for therapeutic intervention with CD83 CAR T.

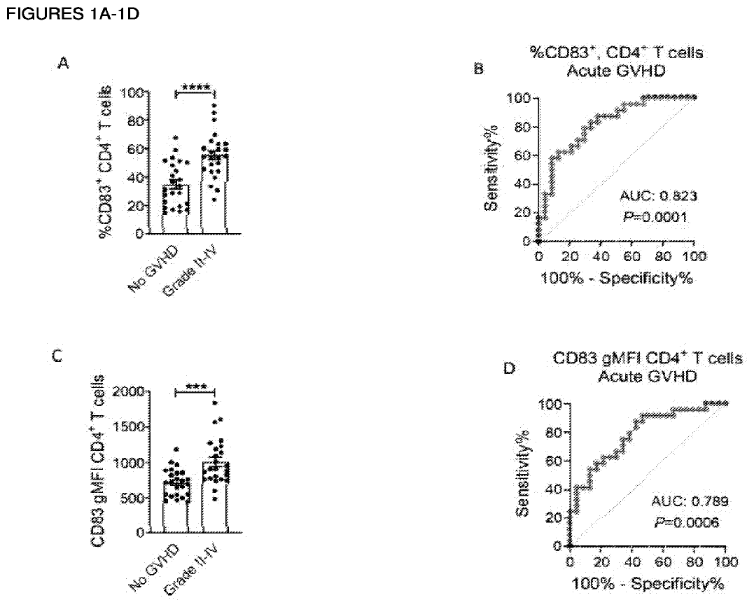

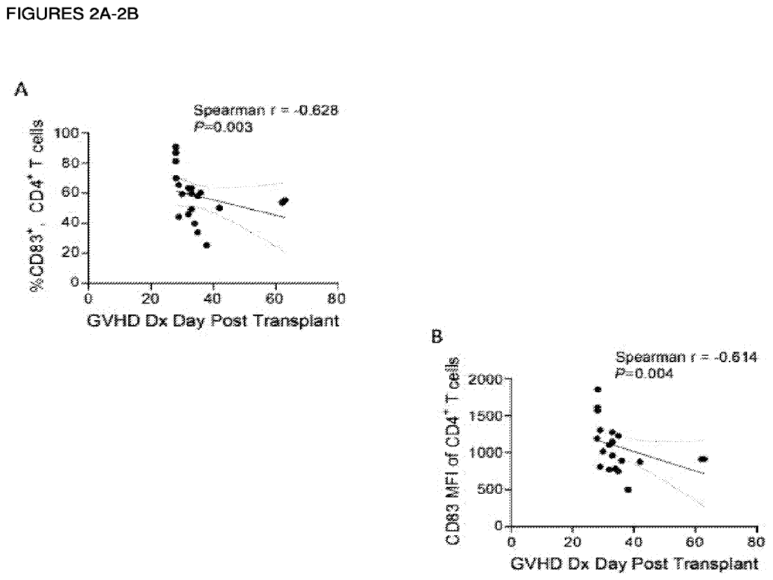

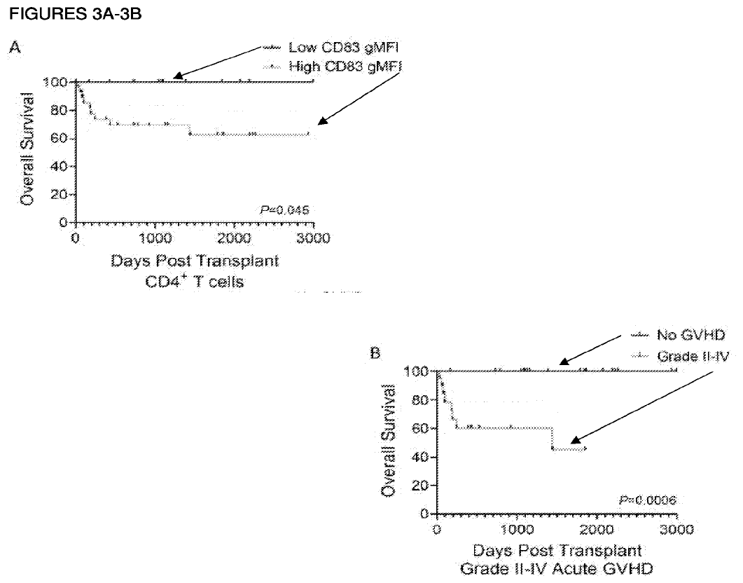

Results/Conclusion: The results of the study are illustrated in FIGS. 1A-1D, 2A-2B, and 3A-3B. The data described herein establish that CD83 is expressed on alloreactive T cells at the time of acute GVHD onset. CD83 expression is negligible on Tregs, anti-leukemia T effectors, or effectors of viral immunity. Anti-CD83 chimeric antigen receptor (CAR) T cells that target CD83 can selectively eliminate pathogenic donor T cells that mediate acute GVHD. The data described herein demonstrate that earlier diagnosis of acute GVHD is associated with even higher CD83 expression. This supports a strategy for administering CD83-targeted therapeutics (e.g., CD83 CAR T cells) which further enhances their potency in the prevention and/or treatment of acute GVHD. Indeed, early, rapid, and comprehensive monitoring of CD83 expression on circulating donor T cells after engraftment can inform the clinical use of CD83 CAR T.

Importantly, high CD83 expression on circulating donor CD4+ T cells before day +100 (geometric mean fluorescence intensity (gMFI)>740.5) is associated with significantly reduced overall survival.

CD83 expression on circulating, donor CD4+ T cells can be used to identify those with acute GVHD and potential risk of death from acute GVHD. Further, targeting CD83-expressing effectors of acute GVHD with CD83 CAR T or CD83-directed therapy offers a selective and effective means of preventing acute GVHD and maintaining graft-versus-leukemia as well as antiviral immunity after alloHCT.

### Example 2: CD83 Expression on B Cells and T Helper Follicular Cells is Increased Upon Chronic GVHD Onset

Chronic GVHD impacts over 60% of alloHCT recipients, effecting skin, muscle, joints, visceral organs, and the immune system. Chronic GVHD is a substantial source of post-transplant morbidity and death, and can emerge years after alloHCT. Chronic GVHD pathogenesis differs from acute GVHD, in that is consists of a triad of allo/autoantibody production, thymic dysfunction, and systemic fibrosis. While several chronic GVHD therapies are now FDA approved (e.g., ruxolitinib, ibrutinib, and belumosudil), many of these agents have nonselective, off-target toxicities that can cause cytopenias and/or infectious complications. Thus, the development of innovative, selective treatments for chronic GVHD is needed.

Methods: To measure CD83 expression on autoreactive B cells and Tfh, peripheral blood (˜50-80 mls) was serially drawn at day +60-100 and then weekly as clinically indicated, or at time of chronic GVHD symptom onset. Peripheral blood mononuclear cells were purified by density gradient and stained for extracellular CD83 expression by flow cytometry. A gMFI cutoff of 396 and 469 was used to determine low versus high CD83 expression for circulating B cells and Tfh, respectively. This threshold can be used as a prognostic indicator for chronic GVHD as well as a cut off for therapeutic intervention with CD83 CAR T.

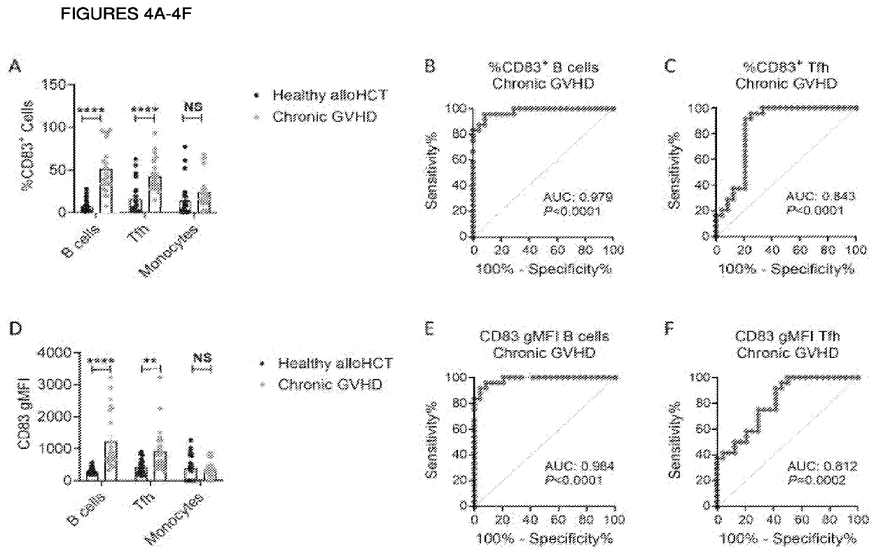

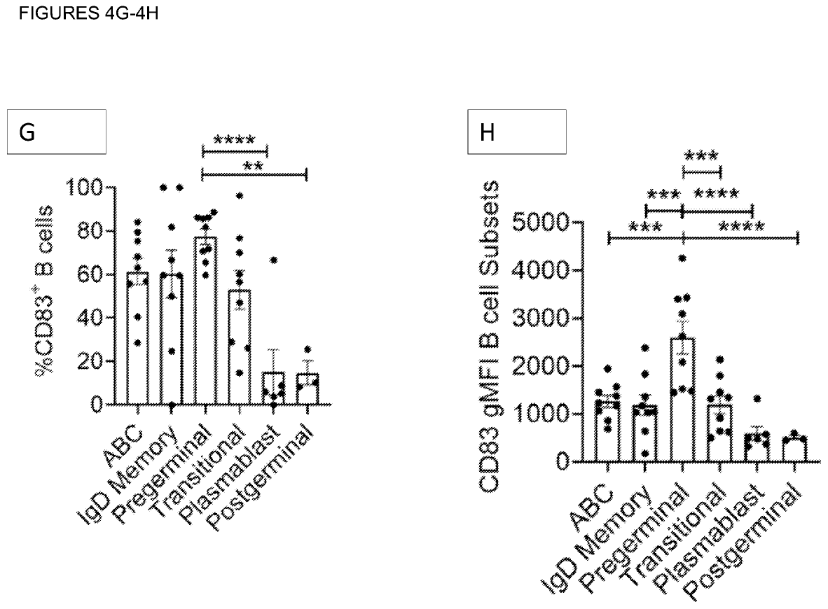

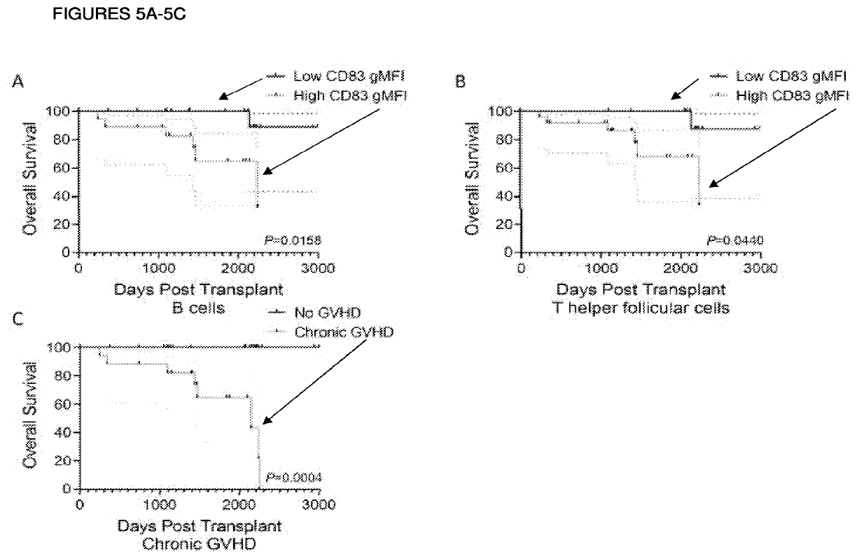

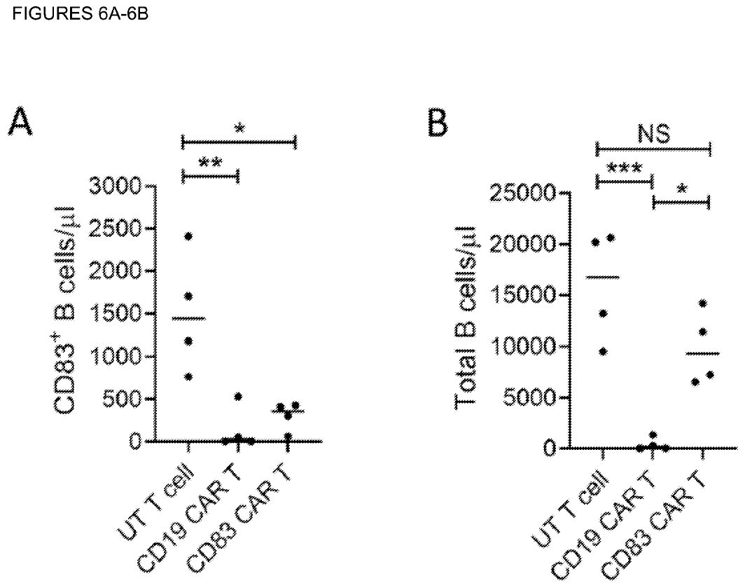

Results/Conclusions: The results of the study are illustrated in FIGS. 4A-4H, 5A-5C, and 6A-6B. CD83 is significantly expressed pathogenic, autoreactive B cells and T helper follicular cells (Tfh), but absent from healthy circulating B cells. This selectivity allows precise eradication of pathogenic effectors of chronic GVHD by using anti CD83 CAR T, yet spare cells needed for humoral and cellular immunity. This is a complete and needed departure from standard pharmacologic agents used to treat chronic GVHD.

A gMFI cutoff of 396 and 469 was used to determine low versus high CD83 expression for circulating B cells and Tfh, respectively. AlloHCT recipients with high CD83 expression on B cells of Tfh after day +100 had a significantly reduced overall survival. Thus, CD83 expression on chronic GVHD effectors offers a therapeutic and prognostic marker of disease, and can inform and guide clinical use of CD83 CAR T.

CD83 expression on circulating, donor B cells and Tfh can be used to identify those with chronic GVHD and potential risk of death from chronic GVHD. Further, targeting CD83 expressing effectors of chronic GVHD with CD83 CAR T or CD83-directed therapy offers a selective and effective means of preventing chronic GVHD and maintaining graft-versus-leukemia as well as antiviral immunity after alloHCT. The data presented herein show CD83-directed therapy, like CD83 CAR T, avoids broad B cell aplasia as observed with CD19 CAR T.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of treating or reducing the risk of developing an alloimmune condition or an autoimmune condition in a subject in need thereof, the method comprising (a) measuring CD83 in a population of immune cells from the subject, and (b) administering to the subject a CD83-targeted therapeutic.

2. The method of claim 1, wherein step (a) comprises measuring CD83 protein levels.

3. The method of claim 2, wherein CD83 protein levels are measured by immunohistochemistry (IHC), flow cytometry, or Western blotting.

4. The method of claim 2, wherein CD83 protein levels are measured via Geometric mean fluorescence intensity (gMFI).

5. The method of claim 1, wherein step (a) comprises measuring CD83 RNA levels.

6. The method of claim 5, wherein CD83 RNA is measured via real time polymerase chain reaction (qRT-PCR) or RNA-Sequencing.

7. The method of any one of claims 1-6, wherein the condition is acute graft-versus-host disease (GvHD) and the population of immune cells in step (a) comprises CD4+ T cells.

8. The method of claim 7, wherein step (a) comprises detecting CD83 expression in at least 40% of CD4+ T cells in the population.

9. The method of claim 7, wherein step (a) comprises measuring CD83 using Geometric mean fluorescence intensity (gMFI) and detecting a gMFI of at least 740.5.

10. The method of any one of claims 7-9, wherein the method is performed within 30 days of the subject receiving allogeneic hematopoietic cell transplantation and prior to the onset of acute GvHD symptoms.

11. The method of any one of claims 1-6, wherein the condition is chronic GvHD and the population of immune cells in step (a) comprises CD19+ B cells and/or T helper follicular cells.

12. The method of claim 11, wherein step (a) comprises detecting CD83 expression in at least 19% of CD19+ B cells in the population.

13. The method of claim 11, wherein step (a) comprises measuring CD19+ B cell CD83 using Geometric mean fluorescence intensity (gMFI) and detecting a gMFI of at least 396.

14. The method of claim 11, wherein step (a) comprises detecting CD83 expression in at least 25% of T helper follicular cells in the population.

15. The method of claim 11, wherein step (a) comprises measuring T helper follicular cell CD83 using Geometric mean fluorescence intensity (gMFI) and detecting a gMFI of at least 469.

16. The method of any one of claims 11-15, wherein the method is performed after 60 days of the subject receiving allogeneic hematopoietic cell transplantation and prior to the onset of chronic GvHD symptoms.

17. The method of any one of claims 1-16, wherein the subject is not suffering from symptoms of GvHD when step (a) is performed.

18. The method of any one of claims 1-6, wherein the condition is systemic lupus erythematosus, multiple sclerosis, sjogren's syndrome, systemic sclerosis/scleroderma, inflammatory bowel disease, or rheumatoid arthritis.

19. The method of any one of claims 1-18, wherein the CD83-targeted therapeutic is an antibody that binds CD83, an antigen-binding antibody fragment that binds CD83, or an antibody-like construct that binds CD83.

20. The method of any one of claims 1-18, wherein the CD83-targeted therapeutic is an immune effector cell genetically modified to express a chimeric antigen receptor (CAR) polypeptide that selectively binds CD83.

21. The method of claim 20, wherein the immune effector cell is a Natural Killer (NK) cell, a cytotoxic T cell, a regulatory T cell, or an innate lymphoid cell (types 1-3).

22. The method of any one of claims 1-21, wherein the method further comprises administering to the subject a second therapeutic agent selected from the group consisting of a corticosteroid, methotrexate, cyclosporine, mycophenolate mofetil, tacrolimus, sirolimus, everolimus, antithymocyte globulin, alemtuzumab, cyclophosphamide, ibrutinib, imatinib, infliximab, etanercept, tocilizumab, alemtuzumab, basiliximab, daclizumab, rituximab, denileukin diftitox, pentostatin, ruxolitinib, belumosudil, abatacept, cyclosporine, thalidomide, halofuginone, hydroxychloroquine, mesenchymal stem cells, type 2 innate lymphoid cells, and regulatory T cells.

23. A method of identifying a subject at risk of developing acute GvHD, the method comprising measuring CD83 in a population of CD4+ T cells from the subject, wherein the presence of CD83 expression in at least 40% of CD4+ T cells in the population or a gMFI of at least 740.5 in the population of CD4+ T cells indicates that the subject is at risk of developing acute GvHD.

24. A method of identifying a subject at risk of developing chronic GvHD, the method comprising measuring CD83 in a population of CD19+ B cells from the subject, wherein the presence of CD83 expression in at least 19% of CD19+ B cells in the population or a gMFI of at least 396 indicates that the subject is at risk of developing chronic GvHD.

25. A method of identifying a subject at risk of developing chronic GvHD, the method comprising measuring CD83 in a population of T helper follicular cells from the subject, wherein the presence of CD83 expression in at least 25% of T helper follicular cells in the population or a gMFI of at least 469 indicates that the subject is at risk of developing chronic GvHD.


SEQUENCE LISTING (71 sequences)

SEQ ID NO: 1 - protein, 12 aa, synthetic construct
  source 1..12: note = VH CDR1 sequence
GFSITTGGYWWT

SEQ ID NO: 2 - protein, 17 aa, synthetic construct
  source 1..17: note = VH CDR2 sequence
GYIFSSGNTNYNPSIKS

SEQ ID NO: 3 - protein, 12 aa, synthetic construct
  source 1..12: note = VH CDR3 sequence
CARAYGKLGFDY

SEQ ID NO: 4 - protein, 12 aa, synthetic construct
  source 1..12: note = VL CDR1 sequence
TLSSQHSTYTIG

SEQ ID NO: 5 - protein, 11 aa, synthetic construct
  source 1..11: note = VL CDR2 sequence
VNSDGSHSKGD

SEQ ID NO: 6 - protein, 9 aa, synthetic construct
  source 1..9: note = VL CDR3 Sequence
GSSDSSGYV

SEQ ID NO: 7 - protein, 5 aa, synthetic construct
  source 1..5: note = VH CDR1 sequence
SDGIS

SEQ ID NO: 8 - protein, 16 aa, synthetic construct
  source 1..16: note = VH CDR2 Sequence
IISSGGNTYYASWAKG

SEQ ID NO: 9 - protein, 7 aa, synthetic construct
  source 1..7: note = VH CDR3 sequence
WGGTYSI

SEQ ID NO: 10 - protein, 13 aa, synthetic construct
  source 1..13: note = VL CDR1 sequence
QSSQSVYNNDFLS

SEQ ID NO: 11 - protein, 7 aa, synthetic construct
  source 1..7: note = VL CDR2 sequence
YASTLAS

SEQ ID NO: 12 - protein, 13 aa, synthetic construct
  source 1..13: note = VL CDR3 sequence
TGTYGNSAWYEDA

SEQ ID NO: 13 - protein, 5 aa, synthetic construct
  source 1..5: note = VH CDR1 sequence
SNAMI

SEQ ID NO: 14 - protein, 16 aa, synthetic construct
  source 1..16: note = VH CDR2 sequence
AMDSNSRTYYATWAKG

SEQ ID NO: 15 - protein, 11 aa, synthetic construct
  source 1..11: note = VH CDR3 sequence
GDGGSSDYTEM

SEQ ID NO: 16 - protein, 13 aa, synthetic construct
  source 1..13: note = VL CDR1 sequence
QSSQSVYGNNELS

SEQ ID NO: 17 - protein, 7 aa, unidentified
  source 1..7: note = VL CDR2 sequence
QASSLAS

SEQ ID NO: 18 - protein, 11 aa, synthetic construct
  source 1..11: note = VL CDR3 Sequence
LGEYSISADNH

SEQ ID NO: 19 - protein, 120 aa, synthetic construct
  source 1..120: note = anti-CD83 scFv VH domain
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTIVIVSS

SEQ ID NO: 20 - protein, 111 aa, synthetic construct
  source 1..111: note = anti-CD83 scFv VL domain (VL-GBM00)
QPVLTQSPSASASLGNSVKITCTISSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 21 - protein, 453 aa, synthetic construct
  source 1..453: note = anti-CD83 scFv VH domain (20D04)
METGLRWLLLVAVLKGVQCQSVEESGGRLVTPGTPLTLTCTVSGFSLSNNAINWVRQAPGKGLEWIGYIWSGGLTYYANWAEGRFTISKTSTTVDLKMTSPTIEDTATYFCARGINNSALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRWSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 22 - protein, 238 aa, synthetic construct
  source 1..238: note = anti-CD83 scFv VL domain (20D04)
MDMRAPTQLLGLLLLWLPGARCADVVMTQTPASVSAAVGGTVTINCQASESISNYLSWYQQKPGQPPKLLIYRTSTLASGVSSRFKGSGSGTEYTLTISGVQCDDVATYYCQCTSGGKFISDGAAFGGGTEWVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 23 - protein, 452 aa, synthetic construct
  source 1..452: note = anti-CD83 scFv VH domain (11G05)
METGLRWLLLVAVLKGVQCQSVEESGGRLVTPGTPLTLTCTVSGFTISDYDLSWVRQAPGEGLKYIGFIAIDGNPYYATWAKGRFTISKTSTTVDLKITAPTTEDTATYFCARGAGDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 24 - protein, 238 aa, synthetic construct
  source 1..238: note = anti-CD83 scFv VL domain
MDTREPTQLLGLLLLWLPGARCADVVMTQTPASVSAAVGGTVTINCQSSKNVYNNNWLSWFQQKPGQPPKLLIYYASTLASGVPSRFRGSGSGTQFTLTISDVQCDDAATYYCAGDYSSSSDNGFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 25 - protein, 453 aa, synthetic construct
  source 1..453: note = anti-CD83 scFv VH domain (14C12)
METGLRWLLLVAVLKGVHCQSVEESGGRLVTPGTPLTLTCTASGFSRSSYDMSWVRQAPGKGLEWVGVISTAYNSHYASWAKGRFTISRTSTTVDLKMTSLTTEDTATYFCARGGSWLDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVWDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 26 - protein, 239 aa, synthetic construct
  source 1..239: note = anti-CD83 scFv VL domain
  VARIANT 3: note = X can be any naturally occurring amino acid
MDXRAPTQLLGLLLLWLPGARCALVMTQTPASVSAAVGGTVTINCQSSQSVYDNDELSWYQQKPGQPPKLLIYALASKLASGVPSRFKGSGSGTQFALTISGVQCDDAATYYCQATHYSSDWYLTFGGGTEVVVKGFPVAPTVLLFPPSSDEVATGTVTIVCVANKYFPDVTVTWEVDGTTQTTGTENSKTPQNSADCTYNLSSTLTLTSTQYNSHKEYTCKVTQGTTSVVQSFSRKNC

SEQ ID NO: 27 - protein, 455 aa, synthetic construct
  source 1..455: note = anti-CD83 scFv VH domain (020B08)
METGLRWLLLVAVLKGVQCQSVEESGGRLVTPGTPLTLCTVSGFSLSSYDMTWVRQAPGKGLEWIGIIYASGTTYYANWAKGRFTISKTSTTVDLKVTSPTIGDTATYFCAREGAGVSMTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 28 - protein, 236 aa, synthetic construct
  source 1..236: note = anti-CD83 scFv VL domain (020B08)
MDMRAPTQLLGLLLLWLPGARCAYDMTQTPASVEVAVGGTVTIKCQASQSISTYLDWYQQKPGQPPKLLIYDASDLASGVPSRFKGSGSGTQFTLTISDLECADAATYYCQQGYTHSNVDNVFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 29 - protein, 459 aa, synthetic construct
  source 1..459: note = anti-CD83 scFv VH domain (006G05)
METGLRWLLLVAVLKGVQCQSVEESGGRLVSPGTPLTLTCTASGFSLSSYDMSWVRQAPGKGLEYIGIISSSGSTYYASWAKGRFTISKTSTTVDLEVTSLTTEDTATYFCSREHAGYSGDTGHLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVGIGPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 30 - protein, 236 aa, synthetic construct
  source 1..236: note = anti-CD83 scFv VL domain (006G05)
MDMRAPTQLLGLLLLWLPGARCAYDMTQTPASVEVAVGGTVAIKCQASQSVSSYLAWYQQKPGQPPKPLIYEASMLAAGVSSRFKGSGSGTDFTLTISDLECDDAATYYCQQGYSISDIDNAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 31 - protein, 454 aa, synthetic construct
  source 1..454: note = anti-CD83 scFv VH domain
METGLRWLLLVAVLKGVQCQSVEESGGRLVTPGTPLTLCTVSGIDLSSDGISWVRQAPGKGLEWIGIISSGGNTYYASWAKGRFTISRTSTTVDLKMTSLTTEDTATYFCARVVGGTYSIWGQGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLICMITDFFPEDITVEWOWNGQPAENYKNIQPIMDTDGSYFVYSKLNVQKSNWEAGNTFTCSVLHEGLHNHHTEKSLSHSPGK

SEQ ID NO: 32 - protein, 239 aa, synthetic construct
  source 1..239: note = anti-CD83 scFv VL domain (96G08)
MDTRAPTQLLGLLLLWLPGATFAQVLTQTASPVSAPVGGTVTINCSSQSVYNNDFLSWYQQKPGQPPKLLIYYASTLASGVPSRFKGSGSGTQFTLTISDLECDDAATYYCTGTYGNSAWYEDAFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 33 - protein, 461 aa, synthetic construct
  source 1..461: note = anti-CD83 scFv VH domain (95F04)
METGLRWLLLVAVLKGVQCQSVEESGGRLVTPGTPLTLTCTVSGIDLSSNAMIWVRQAPREGLEWIGAMDSNSRTYYATWAKGRFTISRTSSITVDLKITSPTTEDTATYFCARGDGGSSDYTEMWGPGTLVTVSSASTKGPSVYPLAPGSAAQTNSMVTLGCLVKGYFPEPVTVTWNSGSLSSGVHTFPAVLQSDLYILSSSVTVPSSTWPSETVTCNVAHPASSTKVDKKIVPRDCGCKPCICTVPEVSSVFIFFPKPKDVLTITLTPKVTCVVVDISKDDPEVQFSWFVDDVEVHTAQTQPREEQFNSTFRSVSELPIMHQDWLNGKEFKCIRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 237 aa, synthetic construct
  source 1..237: note = anti-CD83 scFv VL domain (95F04)
MDTRAPTQLLGLLLLWLPGATFAQAVVTQTTSPVSAPVGGTVTINCQSSQSVYGNNELSWYQQKPGQPPKLLIYQASSLASGVPSRFKGSGSGTQFTLTISDLECDDAATYYCLGEYSISADNHFGGGTEVVVKRTPVAPTVLLFPPSSAELATGTATIVCVANKYFPDGTVTWKVDGITQSSGINNSRTPQNSADCTYNLSSTLTLSDEYNHDEYTCQVAQDSGSPVVQSFSRKSC

SEQ ID NO: 35 - protein, 221 aa, synthetic construct
  source 1..221: note = anti-CD83 scFv VH domain
QVQLVQSGGAVVQPGRSLRLSCAASGFTFSTYGMHWVRQAPGKGLEWVAAVSYDGSNKYYADFVKGRFTISRDNPKNTLYLQMNSLRADDTAVYYCARRGGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 36 - protein, 110 aa, synthetic construct
  source 1..110: note = anti-CD83 scFv VL domain
LTQPPPASGTPGQQRVTISCSGSSSNIGSNTVNWYQQLPGTAPKLLIYYGNDQRPSGVPDRFSASKSGTSASLAISGLQSEDEAHYYCAAWDGSLNGGVIFGGGTKVTLG

SEQ ID NO: 37 - protein, 109 aa, synthetic construct
  source 1..109: note = anti-CD83 scFv VL domain
VTQPPSASGTPGQRVTISCSGSSSNIGTNPVNWYQQLPGTAPKLLIYTTDQRPSGVPDRFSGSKSGTSASLAISGLQSEDEADYYCAAWDDSLSGLYVFGTGTKVTVLG

SEQ ID NO: 38 - protein, 109 aa, synthetic construct
  source 1..109: note = anti-CD83 scFv VL domain
MTHTPLSLSVTPGQPASISCKSSQSLLHSDGKTYLYWYLQRPGQSPQPLIYEVSNRFSGVPDRFSGSGSGTDFTLKISRVQAEDVGVYYCMQSLQLWFTGQGTKVEIKR

SEQ ID NO: 39 - protein, 110 aa, synthetic construct
  source 1..110: note = anti-CD83 scFv VL domain
MTQSPLSLPVTLGQPASISCRSSGSLIHSDGNTYLDWFQQRPGQSPRRLIYKVSNRDSGVPDRFSGSGSGTDFTLRISRVEAEDIGVYYCMQATHWPRTFGQGTKVEIKR

SEQ ID NO: 40 - protein, 110 aa, synthetic construct
  source 1..110: note = anti-CD83 scFv VL domain
MTQSPLSLPVTLGQPASISCRSSQSLVDSAGNTFLHWFHQRPGQSPRRLIYKVSNRDSGVPDRFSGSGSGTDFTLKISRVEAEDVGVYYCMQGTHWPRTFGQGTKVEIKR

SEQ ID NO: 41 - protein, 110 aa, synthetic construct
  source 1..110: note = anti-CD83 scFv VL domain
LTQSPLSLPVTLGQPASISCKSSQSLVDSDGNTYLNWFQQRPGQSPRRLIYKVSNRDSGVPDRFSGSGSGTDFTLKISRVEAEDVGVYYCMQGTHWPRTFGQGTKVEIKR

SEQ ID NO: 42 - protein, 110 aa, synthetic construct
  source 1..110: note = anti-CD83 scFv VL domain
MTQSPLSLPVTLGQPASISCRSSQSLVHSDGNMYLNWFQQRPGQSPRRLIYKVSNRDSGVPDRFSGSGSGTDFTLKISRVEAEDVGVYYCMQATQPTWTFGQGTKLEIKR

SEQ ID NO: 43 - protein, 92 aa, synthetic construct
  source 1..92: note = anti-CD83 scFv VL domain
MTQSPSSLSASVGDRVTITCQASQDISNYLNWYQQKPGKAPKLLIYDASNLETGVPSRFSGSGSGTDFTFTISSATYYCQQTYQGTKLEIKR

SEQ ID NO: 44 - protein, 105 aa, synthetic construct
  source 1..105: note = anti-CD83 scFv VL domain
MTQSPSSLSASVGHPVTITCRASQSLISYLNWYHQKPGKAFKLLIYAASILQSGVPSRFSGSGSGTDFTLTISSLQPENFASYYCQHTDSFPRTFGHGTKVEIKR

SEQ ID NO: 45 - protein, 108 aa, synthetic construct
  source 1..108: note = anti-CD83 scFv VL domain
LTQPPSASGTPGQGVTISCRGSTSNIGNNVVNWYQHVPGSAPKLLIWSNIQRPSGIPDRFSGSKSGTSASLAISGLQSEDQAVYYCAVWDDGLAGWVFGGGTTVTVLS

SEQ ID NO: 46 - protein, 105 aa, synthetic construct
  source 1..105: note = anti-CD83 scFv VL domain
MTQAPVVSVALEQTVRITCQGDSLAIYYDFWYQHKPGQAPVLVIYGKNNRPSGIPHRFSGSSSNTDSLTITGAQAEDEADYYCNSRDSSGNHWVFGGGTNLTVLG

SEQ ID NO: 47 - protein, 111 aa, synthetic construct
  source 1..111: note = anti-CD83 scFv VL domain
LTQSPLSLPVTLGQPASISCKSNQSLVHSDGNTYLNWFQQRPGQSPRRLIYKVSNRDSGVPDRFSGSGSGTDFTLKINRVEAEDVGVYYCMQGTQWPRTFGGQGTKLDIKR

SEQ ID NO: 48 - protein, 119 aa, synthetic construct
  source 1..119: note = anti-CD83 scFv VH domain (VH-GBM01)
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 49 - protein, 119 aa, synthetic construct
  source 1..119: note = anti-CD83 scFv VH domain (VH-GBM02)
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSS

SEQ ID NO: 50 - protein, 119 aa, synthetic construct
  source 1..119: note = anti-CD83 scFv VH domain (VH-GBM03)
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 51 - protein, 119 aa, synthetic construct
  source 1..119: note = anti-CD83 scFv VH domain (VH-GBM04)
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSS

SEQ ID NO: 52 - protein, 119 aa, synthetic construct
  source 1..119: note = anti-CD83 scFv VH domain (VH-GBM05)
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSS

SEQ ID NO: 53 - protein, 120 aa, synthetic construct
  source 1..120: note = anti-CD83 scFv VH domain (VH-GBM06)
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSS

SEQ ID NO: 54 - protein, 111 aa, synthetic construct
  source 1..111: note = anti-CD83 scFv VL domain (VH-GBM01)
QLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 55 - protein, 111 aa, synthetic construct
  source 1..111: note = anti-CD83 scFv VL domain (VH-GBM02)
LPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 56 - protein, 15 aa, synthetic construct
  source 1..15: note = Linker sequence
GGGGSGGGGSGGGGS

SEQ ID NO: 57 - protein, 247 aa, synthetic construct
  source 1..247: note = anti-CD83 scFv
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAASSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAA

SEQ ID NO: 58 - protein, 253 aa, synthetic construct
  source 1..253: note = anti-CD83 scFv
QVQLKESGPGLVKPSQSLSLTCSVTGFSITTGGYWWTWIRQFPGQKLEWMGYIFSSGNTNYNPSIKSRISITRDTSKNOFFLQLNSVTTEGDTARYYCARAYGKLGFDYWGQGTLVTVSSGGGGSGGGGSGGGGSQVQLKESGPGINKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTV

SEQ ID NO: 59 - protein, 245 aa, synthetic construct
  source 1..245: note = anti-CD83 scFv
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 60 - protein, 245 aa, synthetic construct
  source 1..245: note = anti-CD83 scFv
QVQLQESGPGINKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 61 - protein, 245 aa, synthetic construct
  source 1..245: note = anti-CD83 scFv
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 62 - protein, 244 aa, synthetic construct
  source 1..244: note = anti-CD83 scFv
QVQLQESGPGINKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLSEDEADYYCGSSDSSGYVFGSGTKVTVL

SEQ ID NO: 63 - protein, 245 aa, synthetic construct
  source 1..245: note = anti-CD83 scFv
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSQLVLTQSPSASASLGASVKLICTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 64 - protein, 246 aa, synthetic construct
  source 1..246: note = anti-CD83 scFv
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 65 - protein, 245 aa, synthetic construct
  source 1..245: note = anti-CD83 scFv
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQFPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 66 - protein, 245 aa, synthetic construct
  source 1..245: note = anti-CD83 scFv
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLIFSNLQSDDEAEYHCGSSDSSGYVFGSGTKVTVL

SEQ ID NO: 67 - protein, 245 aa, synthetic construct
  source 1..245: note = anti-CD83 scFv
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 68 - protein, 246 aa, synthetic construct
  source 1..246: note = anti-CD83 scFv
QVQLQESGPGLVKPSETLSLTCTVSGFSITTGGYVWVTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSLPVLTQFPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 69 - protein, 245 aa, synthetic construct
  source 1..245: note = anti-CD83 scFv
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 70 - protein, 246 aa, synthetic construct
  source 1..246: note = anti-CD83 scFv
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 71 - protein, 246 aa, synthetic construct
  source 1..246: note = anti-CD83 scFv
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTISSQHSTYTIGWYQQHPDKAPKYVMYVNSDGSHSKGDGIPDRFSGSSSGAHRYLSISNIQPEDEADYFCGSSDSSGYVFGSGTQLTVL